(19) United States
(12) Patent Application Publication
(10) Pub. No.: US 2012/0144516 A1
(43) Pub. Date: Jun. 7, 2012

(54) TRANSGENIC SUGAR BEET EVENT GM RZ13

(75) Inventors: Stig Lennart Tuvesson, Teckomatorp (SE); Johannes Jacobus Ludgerus Gielen, Bouloc (FR); Signe Irene Elisabet Wremert Weich, Landskrone (SE); Jan Hugo Benesfelt, Landskrone (SE); Britt-Louise Lennefors, Eslov (SE); Petronella Maria Van Roggen, Landskrone (SE)

(73) Assignee: SYNGENTA PARTICIPATIONS AG, Basel (CH)

(21) Appl. No.: 13/140,878

(22) PCT Filed: Dec. 16, 2009
(86) PCT No.: PCT/EP2009/067251

§ 371 Date: Feb. 24, 2012

(30) Foreign Application Priority Data
Dec. 19, 2008 (EP) EP 08172387.6

(51) Int. Cl. A01H 5/00; C07H 21/04; C12Q 1/68; C10L 1/00; A01H 5/10; A01H 1/02; C07C 31/08; C07C 31/12; C12N 15/11; G01N 33/53

(52) U.S. Cl. 800/265; 536/23.1; 536/24.33; 435/6.1; 435/6.12; 436/501; 536/24.3; 800/301; 568/840; 44/307

(57) ABSTRACT

A novel transgenic sugar beet event designated GM RZ13 is disclosed. The invention relates to nucleic acids that are unique to event GM RZ13. The invention also relates to assays for detecting the presence of the GM RZ13 event based on DNA sequences of the recombinant constructs inserted into the sugar beet genome that resulted in the GM RZ13 event and of genomic sequences flanking the insertion site. The invention further relates to sugar beet plants comprising the genotype of GM RZ13 and to methods for producing a sugar beet plant by crossing a sugar beet plant comprising the GM RZ13 genotype with itself or another sugar beet variety. Seeds of sugar beet plants comprising the GM RZ13 genotype are also objects of the present invention.

### FIELD OF THE INVENTION

The invention relates to a novel transgenic sugar beet event designated GM RZ13 and nucleic acids that are unique to event GM RZ13. The invention also relates to assays for detecting the presence of the GM RZ13 event based on DNA sequences of the recombinant constructs inserted into the sugar beet genome that resulted in the GM RZ13 event and of genomic sequences flanking the insertion site. Embodiments of the invention further provide for sugar beet plants comprising the genotype of GM RZ13 which are resistant to Beet Necrotic Yellow Vein Virus (BNYVV) and for methods for producing a sugar beet plant by crossing a sugar beet plant comprising the GM RZ13 genotype with itself or another sugar beet variety. Seeds of sugar beet plants comprising the GM RZ13 genotype are also objects of the present invention.

### BACKGROUND OF THE INVENTION

The present patent application relates generally to the field of plant molecular biology, plant transformation, and plant breeding. More specifically, the application relates to disease resistant transgenic sugar beet plants comprising a novel transgenic genotype and to methods of detecting the presence of the sugar beet plant DNA in a sample and compositions thereof.

Beet Necrotic Yellow Vein Virus (BNYVV) is the causal agent of rhizomania (Tamada and Baba, 1973), one of the most important sugar beet diseases worldwide with a potential yield reduction up to about 90% in susceptible varieties (Johansson, 1985). Infected sugar beet plants further show a reduction of sugar content in the taproot from about 17% to about 11%. Rhizomania was first described in Italy in the 1950s (Canova, 1959), but is now reported from more than 80% of the sugar beet growing areas around the world (Lennefors et al., 2005). BNYVV is transmitted by Polymyxa betae (Tamada, 1975), a soilborne protoctist with resting spores that can survive for more than 15 years in the soil (Abe and Tamada, 1986).

Characteristic symptoms of rhizomania (“root madness”) infections in sugar beets are yellowing, stunting, small taproots and an increased number of fibrous roots. The vascular tissues of the taproot show a light brown discoloration. At rare occasions the virus spreads systemically to the leaves resulting in formation of necrotic vein yellowing, but normally the virus remains confined to the root tissues (Johansson, 1985; Tamada, 1975).

Different genetic strains or isolates of BNYVV have been identified by restriction fragment length polymorphism or single strand conformation polymorphism analyses (Kruse et al., 1994; Koenig et al., 1995). The major groups of the European BNYVV isolates have been named A, B and P, of which type A is most widespread. Type A and B comprise four single-stranded genomic RNAs, whereas type P contains also a fifth RNA species. The P-type is considered to be more pathogenic or virulent than the A and B types (Heijbroek et al., 1999).

Since other strategies like, for example, biological control or mineral fertilizers have not provided sufficient resistance levels or require the use of fungicides or fumigants, the introgression of resistance into sugar beet cultivars is generally considered essential to ensure economically profitable sugar beet production in soils infested with rhizomania. As a consequence of the spread of rhizomania, sugar beet breeding companies have bred intensively for more than 20 years to develop rhizomania resistant varieties. The first rhizomania resistant variety available on the market was called “Rizor”, originating from Italian germplasm (Biancardi et al., 2002; De Biaggi, 1987). Most commercial rhizomania resistant varieties known today, however, originate from the Holly source (Lewellen et al., 1987), where the major dominant gene Rz1 confers resistance to BNYVV (Pelsy and Merdinoglu, 1996; Scholten et al., 1996). Today, this conventional resistance is present in about 90% of rhizomania resistant sugar beet varieties. Other BNYVV resistant sources are WB41 and WB42 originating from two plants of Beta vulgaris ssp maritime collected in Denmark (Lewellen et al. 1987; Whitney, 1989). The rhizomania resistant sugar beet line C48 was developed from crosses of WB41 and WB42 to line C37 (Lewellen and Whitney, 1993). The resistance from line C48 is nowadays combined with the Holly resistance in several varieties. Other sources of Rhizomania resistance are WB151, WB169, C28, and C50 (Lewellen, 1995).

However, the resistance in BNYVV resistant sugar beet plants containing resistances obtained from conventional sources seems to be only partial as the plants get infected by the virus which then starts to multiply. Although the virus multiplies at a lower level than in susceptible genotypes, the virus multiplication still allows the virus to spread. Further, with increasing infestation levels of the virus, there is a risk that the conventional sources do not result in a sufficient resistance.

In addition, breeding for rhizomania resistance is limited by the durability of conventional resistances in the germplasm pool of sugar beet. In fact, strong rhizomania symptoms caused by highly pathogenic deviating strains of BNYVV were observed in rhizomania resistant varieties based on the Holly source (Liu et al., 2005). These highly pathogenic deviating strains of BNYVV have been detected in several locations in the USA and in Europe during the last few years. It is highly likely (and has even already been observed in the fields) that the partial resistance conferred by the conventional resistances (like Holly or C48) is broken by these highly pathogenic deviating strains of BNYVV.

In contrast to the introgression of genes from conventional sources of resistance, the transgenic expression of virus-derived sequences offers an alternative solution to combat viral diseases, a strategy known as pathogen-derived resistance. The concept of parasite- or pathogen-derived resistance was coined by Sanford and Johnston (1985) and was demonstrated for the first time in transgenic tobacco plants (Nicotiana tabacum) expressing the coat protein gene from Tobacco Mosaic Virus (Powell-Abel et al., 1986). Since then, it has been reported to function in many different crops and against a large number of viruses (Kaniewski and Lawson, 1998). Besides using coat protein genes, resistance has also been achieved in plants expressing other viral sequences like, for example, replicase or dysfunctional movement proteins (Baulcombe, 1996). The discovery that plants possess an innate defense system against viral invaders based on the sequence-specific degradation of foreign or aberrant RNAs, referred to as post-transcriptional gene silencing, was widely used and proved to be instrumental for engineering virus resistance and greatly improved the feasibility of the transgenic expression of virus-derived sequences (Waterhouse et al., 1999; Voinnet, 2001).

The mechanisms underlying transgenic resistance to virus infections have been subject to speculation. The observation, however, that transgenic virus resistance and post-transcriptional gene silencing (PTGS) in some cases share many characteristics has provided novel insights and opportunities for engineering virus resistance in plants (Waterhouse et al., 2001; Tenllado et al., 2004). Transformation of tobacco with a construct consisting of an inverted repeat of viral sequences derived from Potato Virus Y leading to the transgenic expression of a dsRNA was shown to confer strong levels of resistance at exceptionally high frequencies (Waterhouse et al., 1998). It would thus be advantageous to be able to provide resistance against BNYVV in sugar beet by transformation of sugar beet with a construct consisting of an inverted repeat of viral sequences derived from BNYVV leading to the transgenic expression of a dsRNA to confer strong levels of resistance against the virus.

The expression of foreign genes in plants can be influenced by their chromosomal position, perhaps due to chromatin structure or the proximity of transcriptional regulation elements close to the integration site (See, for example, Weising et al., 1988, “Foreign Genes in Plants,” Ann. Rev. Genet. 22:421-477). For this reason, it is often necessary to screen a large number of events in order to identify an event characterized by optimal expression of an introduced gene of interest. For example, it has been observed in plants and in other organisms that there may be wide variations in levels of expression of a heterologous gene introduced into the chromosome of a plants' genome among individually selected events. There may also be differences in spatial or temporal patterns of expression, for example, differences in the relative expression of a transgene in various plant tissues, that may not correspond to the patterns expected from transcriptional regulatory elements present in the introduced gene construct. An event that has desired levels or patterns of transgene expression is useful for introgressing the transgene into other genetic backgrounds by sexual out-crossing using conventional breeding methods. Progeny of such crosses maintain the transgene expression characteristics of the original transformant. This strategy is used to ensure reliable gene expression in a number of varieties that are well adapted to local growing conditions.

It would thus be also advantageous to be able to detect the presence of a particular event in a plant in order to determine whether progeny of a sexual cross contain a transgene of interest. In addition, a method for detecting a particular event would be helpful for complying with regulations requiring the pre-market approval and labeling of foods derived from recombinant crop plants, for example. It is possible to detect the presence of a transgene by any well-known nucleic acid detection method including, but not limited to thermal amplification (polymerase chain reaction (PCR)) using polynucleotide primers or DNA hybridization using nucleic acid probes. Typically, for the sake of simplicity and uniformity of reagents and methodologies for use in detecting a particular DNA construct that has been used for transforming various plant varieties, these detection methods generally focus on frequently used genetic elements, for example, promoters, terminators, and marker genes, because for many DNA constructs, the coding sequence region is interchangeable. As a result, such methods may not be useful for discriminating between constructs that differ only with reference to the coding sequence. In addition, such methods may not be useful for discriminating between different events, particularly those produced using the same DNA construct unless the sequence of chromosomal DNA adjacent to the inserted heterologous DNA (“flanking DNA”) is known.

For the foregoing reasons, there is a need for a BNYVV resistant transgenic sugar beet event showing a strong resistance to BNYVV that is superior to the resistance obtained from conventional sources and shows stable resistance against the highly pathogenic deviating strains of BNYVV observed recently. Said BNYVV resistant transgenic sugar beet event comprises novel nucleic acid sequences which are unique to the transgenic sugar beet event, useful for identifying the transgenic sugar beet event and for detecting nucleic acids from the transgenic sugar beet event in a biological sample.

### SUMMARY OF THE INVENTION

The present invention relates to transformed sugar beet (Beta vulgaris L.), designated GM RZ13 (or SBVR111, a designation that is used interchangeably with the designation GM RZ13), comprising a novel transgenic genotype that comprises an inverted repeat comprising a part of the RNA-1 gene transcript of the BNYVV. This portion of RNA-1 of BNYVV encodes the RNA dependent RNA polymerase (RdRp) or replicase gene. The GM RZ13 event also comprises the manA gene (also known as pmi) from Escherichia coli encoding a phosphomannose isomerase (PMI) protein that confers upon sugar beet cells the ability to utilize mannose as a carbon source. The event GM RZ13 is also known as SBVR111. The invention also provides transgenic sugar beet plants comprising the genotype of the invention, seed from transgenic sugar beet plants comprising the genotype of the invention, and to methods for producing a transgenic sugar beet plant (e.g., a hybrid plant) comprising the genotype of the invention by crossing a sugar beet inbred comprising the genotype of the invention with itself or another sugar beet line of a different genotype. The transgenic sugar beet plants of the invention may have essentially all of the morphological and physiological characteristics of the corresponding isogenic non-transgenic sugar beet plant in addition to those conferred upon the sugar beet plant by the novel genotype of the invention.

European patent No. EP 1 169 463 describes the use of sequences of between 15 nucleotides and up to 6746 nucleotides of genomic RNA1 of Beet Necrotic Yellow Vein Virus (BNYVV) in an antisense approach to convey resistance to BNYVV to a sugar beet plant, However, the patent does not provide any data of field trials and specifically no data of field trials on soils being infected with the different strains of BNYVV, in particular with the new and the aggressive and highly pathogenic deviating BNYVV strains. On the other hand, however, the specific event of the present invention provides a consistent and strong reduction of the virus titer of all types of BNYVV compared to conventional resistances and further a strong control of the new highly pathogenic BNYVV strain.

The publication of Lennefors et al. (2006), which is related to dsRNA mediated resistance to BNAVV infections in sugar beet, describes the transformation of sugar beet using the construct of the present invention and BNYVV resistant sugar beet obtained by that transformation. This publication, however, does not provide any specific information regarding the specific BNYVV resistant event of the present invention and thus does not allow the skilled person to obtain same.

The present invention also provides compositions and methods for detecting the presence of nucleic acids from event GM RZ13 based on the DNA sequence of the inverted repeat comprising a fragment from the BNYVV replicase gene inserted into the sugar beet genome that resulted in the GM RZ13 event and of genomic sequences flanking the insertion site. The GM RZ13 event can be further characterized by analyzing expression levels of PMI proteins as well as by testing efficacy against BNYVV.

According to one aspect, the present invention provides a nucleic acid molecule, particularly an isolated nucleic acid molecule, comprising a nucleotide sequence that is unique to event GM RZ13. In further embodiments, the present invention provides a nucleic acid molecule, preferably an isolated nucleic acid molecule, comprising at least 10 contiguous nucleotides of a heterologous DNA sequence inserted into the sugar beet plant genome of sugar beet event GM RZ13 and at least 10 contiguous nucleotides of a sugar beet plant genome DNA flanking the point of insertion of a heterologous DNA sequence inserted into the sugar beet plant genome of sugar beet event GM RZ13. In some embodiments of this aspect, the nucleic acid molecule according to this aspect may comprise at least 15, 20, 25, 30, 35, 40, 45, or at least 50 contiguous nucleotides of a heterologous DNA sequence inserted into the sugar beet plant genome of sugar beet event GM RZ13 and at least 15, 20, 25, 30, 35, 40, 45, or at least 50 contiguous nucleotides of a sugar beet plant genome DNA flanking the point of insertion of a heterologous DNA sequence inserted into the sugar beet plant genome of sugar beet event GM RZ13. It is to be understood that the term “at least x nucleotides” encompasses nucleic acid molecules having any numerical value starting with x and above. For example, the term “at least 15 nucleotides” is intended to encompass nucleic acid molecules with 15, 16, 17, 18, 19, 20, and more nucleotides. In a further embodiment of this aspect, said nucleic acid molecule, particularly in isolated form, comprises as a nucleotide sequence that is unique to event GM RZ13 a nucleotide sequence selected from the group consisting of SEQ ID NO: 1, SEQ ID NO: 2, SEQ ID NO: 7, SEQ ID NO: 8, and the complements thereof. In yet another embodiment of this aspect, said nucleic acid molecule is comprised in a sugar beet seed deposited at the NCIMB in Aberdeen, Scotland, under the accession No. 41601.

According to another aspect, the present invention provides a nucleic acid molecule, particularly an isolated nucleic acid molecule, comprising a nucleotide sequence that comprises at least one junction sequence of event GM RZ13. A junction sequence spans the junction between the heterologous DNA comprising the inverted repeat comprising a fragment from the BNYVV replicase gene inserted into the sugar beet genome and DNA from the sugar beet genome flanking the insertion site and is diagnostic for the GM RZ13 event. In one embodiment of this aspect, the junction sequence is selected from the group consisting of SEQ ID NO: 1, SEQ ID NO: 7, and the complements thereof.

According to another aspect, the present invention provides a nucleic acid, particularly an isolated nucleic acid, linking a heterologous DNA molecule to the sugar beet plant genome in sugar beet event GM RZ13 comprising a sequence of from about 11 to about 20 contiguous nucleotides. It is to be understood that the length of said isolated nucleic acid can be of any numerical value within this range. In one embodiment of this aspect, the nucleic acid is selected from the group consisting of SEQ ID NO: 1, SEQ ID NO: 7, and the complements thereof.

According to another aspect of the invention, an amplicon comprising a nucleic acid molecule of the invention is provided. In one embodiment of this aspect, the amplicon comprises a nucleic acid molecule of the present invention selected from the group consisting of SEQ ID NO: 1, SEQ ID NO: 2, SEQ ID NO: 3, SEQ ID NO: 7, SEQ ID NO: 8, SEQ ID NO: 9 and the complements thereof.

According to still another aspect of the invention, flanking sequence primers for detecting event GM RZ13 are provided. Such flanking sequence primers comprise a nucleotide sequence of at least 10 to 15 contiguous nucleotides from the 5′ or 3′ flanking sequence. Again, it is to be understood that the length of said flanking sequence primers can be of any numerical value within this range. In one embodiment of this aspect, the contiguous nucleotides are selected from nucleotides 1-237 (inclusive) of SEQ ID NO: 8 (arbitrarily designated herein as the 5′ flanking sequence, this sequence is depicted herein as SEQ ID NO: 9), or the complements thereof. In another embodiment of this aspect, the contiguous nucleotides are selected from nucleotides 1-347 (inclusive) of SEQ ID NO: 2 (arbitrarily designated herein as the 3′ flanking sequence, this sequence is depicted herein as SEQ ID NO: 3), or the complements thereof.

According to another aspect, the present invention provides a pair of polynucleotide primers comprising a first polynucleotide primer and a second polynucleotide primer that function together in the presence of a sugar beet event GM RZ13 DNA template in a sample to produce an amplicon diagnostic for event GM RZ13. In one embodiment of this aspect, one the primer sequence is or is complementary to a sugar beet plant genome sequence flanking the point of insertion of a heterologous DNA sequence inserted into the sugar beet plant genome of sugar beet event GM RZ13, and the other polynucleotide primer sequence is or is complementary to the heterologous DNA sequence inserted into the sugar beet plant genome of the sugar beet event GM RZ13. In one embodiment of this aspect, one of the primer sequences is chosen from SEQ ID NO: 2, SEQ ID NO:3, SEQ ID NO: 8 or SEQ ID NO: 9. In another embodiment of this aspect, the first polynucleotide primer comprises at least 10 contiguous nucleotides from SEQ ID NO:3 or from position 461-807 of SEQ ID NO: 2, or the complements thereof, or comprises at least 10 contiguous nucleotides from SEQ ID NO: 9 or from position 1-237 of SEQ ID NO: 8, and the complements thereof. In a preferred embodiment, said first polynucleotide primer comprises a nucleotide sequence selected from the group consisting of SEQ ID NO: 12, SEQ ID NO: 13, SEQ ID NO: 14, SEQ ID NO:15, SEQ ID NO:16, SEQ ID NO:23, SEQ ID NO:24, SEQ ID NO:25, and the complements thereof. In another embodiment of this aspect, the second polynucleotide primer comprises at least 10 contiguous nucleotides derived from position 1-460 as set forth as SEQ ID NO: 2 or derived from position 238-484 as set forth as SEQ ID NO: 8, or the complements thereof. In still another embodiment of this aspect, the second polynucleotide primer is selected from the group consisting of SEQ ID NO: 4, SEQ ID NO: 5, SEQ ID NO: 6, SEQ ID NO: 10, SEQ ID NO: 18, SEQ ID NO: 19, SEQ ID NO: 20, SEQ ID NO: 26, and complements thereof. In another embodiment of this aspect, the pair of primers is selected from the group of primer pairs consisting of: (a) the polynucleotide primer as set forth as SEQ ID NO: 13 as the first polynucleotide primer and the second polynucleotide primer as set forth as SEQ ID NO: 18, and complements thereof; (b) the polynucleotide primer as set forth as SEQ ID NO: 14 as the first polynucleotide primer and the second polynucleotide primer as set forth as either SEQ ID NO: 10 or SEQ ID NO: 18, and complements thereof; (c) the polynucleotide primer as set forth as SEQ ID NO: 15 as the first polynucleotide primer and the second polynucleotide primer as set forth as SEQ ID NO: 18, and complements thereof; (d) the polynucleotide primer as set forth as SEQ ID NO: 16 as the first polynucleotide primer and the second polynucleotide primer as set forth as SEQ ID NO: 18, and complements thereof; (e) the polynucleotide primer as set forth as SEQ ID NO: 12 as the first polynucleotide primer and the second polynucleotide primer as set forth as either SEQ ID NO: 19, SEQ ID NO: 4, SEQ ID NO: 5, or SEQ ID NO: 6, and complements thereof; (f) the polynucleotide primer as set forth as SEQ ID NO: 19 as the first polynucleotide primer and the second polynucleotide primer as set forth as SEQ ID NO: 24, and complements thereof; (g) the polynucleotide primer as set forth as SEQ ID NO: 20 as the first polynucleotide primer and the second polynucleotide primer as set forth as SEQ ID NO: 24, and complements thereof; and (h) the polynucleotide primer as set forth as SEQ ID NO: 25 as the first polynucleotide primer and the second polynucleotide primer as set forth as SEQ ID NO: 26, and complements thereof. In yet another embodiment of this aspect, the pair of primers is selected from the group of primer pairs consisting of: (a) the polynucleotide primer as set forth as SEQ ID NO: 13 as the first polynucleotide primer and the second polynucleotide primer as set forth as either SEQ ID NO: 11 or SEQ ID NO: 17, and complements thereof; (b) the polynucleotide primer as set forth as SEQ ID NO: 12 as the first polynucleotide primer and the second polynucleotide primer as set forth as either SEQ ID NO: 21 or SEQ ID NO: 22, and complements thereof; the polynucleotide primer as set forth as SEQ ID NO: 19 as the first polynucleotide primer and the second polynucleotide primer as set forth as SEQ ID NO: 23, and complements thereof; and the polynucleotide primer as set forth as SEQ ID NO: 20 as the first polynucleotide primer and the second polynucleotide primer as set forth as SEQ ID NO: 23, and complements thereof.

According to another aspect of the invention, methods of detecting the presence of DNA unique to event GM RZ13 in a biological sample are provided. Such methods comprise: (a) contacting the sample comprising DNA with a pair of primers that, when used in a nucleic-acid amplification reaction with genomic DNA from sugar beet event GM RZ13, produces an amplicon that is diagnostic for sugar beet event GM RZ13; (b) performing a nucleic acid amplification reaction, thereby producing the amplicon; and (c) detecting the amplicon. In a preferred embodiment, the pair of primers used in the above method is one of the primer pairs of the present invention mentioned above. In a further preferred embodiment, said method of detecting the presence of DNA unique to event GM RZ13 in a biological sample is either a gel-based assay comprising the steps of (i) contacting the sample comprising sugar beet nucleic acids with a pair of primers having the sequence as set forth as SEQ ID NOs: 5 and 12, or a pair of primers having the sequence as set forth as SEQ ID NOs: 13 and 18; (ii) performing a nucleic acid amplification reaction, thereby producing the amplicon; and (iii) detecting the amplicon; or a TaqMan® assay comprising the steps of (i) contacting the sample comprising sugar beet nucleic acids with a pair of primers having the sequence as set forth as SEQ ID NOs: 25 and 26 and a TaqMan® probe having the sequence as set forth as SEQ ID NO: 27; (ii) performing a nucleic acid amplification reaction, thereby producing the amplicon; and (iii) detecting the increase in fluorescence emitted by the reporter dye cleaved from the probe and separated from the quencher dye during the amplification in step (ii). In another embodiment, such a method comprise: (a) contacting the sample with a probe that hybridizes under high stringency conditions with genomic DNA from event GM RZ13 and does not hybridize under high stringency conditions with DNA of a control sugar beet plant; (b) subjecting the sample and probe to high stringency hybridization conditions; and (c) detecting hybridization of the probe to the nucleic acid molecule. In further embodiment of this aspect, the amplicon or the probe comprises the nucleotide sequence derived from the group consisting of SEQ ID NO: 1, SEQ ID NO: 2, SEQ ID NO: 3, SEQ ID NO: 7, SEQ ID NO: 8, SEQ ID NO: 9 and complements thereof.

According to another aspect of the invention, a kit is provided for the detection of nucleic acids that are unique to event GM RZ13 in a biological sample. The kit includes at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event GM RZ13 in the sample. The kit further includes other materials necessary to enable nucleic acid hybridization or amplification methods. In one embodiment of this aspect, a nucleic acid molecule contained in the kit comprises a nucleotide sequence selected from the group consisting of SEQ ID NO: 1, SEQ ID NO: 4, SEQ ID NO: 5, SEQ ID NO: 6, SEQ ID NO: 7, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and the complements thereof.

The present invention further provides a sugar beet plant comprising the transgenic genotype of the invention, wherein the transgenic genotype confers upon the sugar beet plant resistance to Beet Necrotic Yellow Vein Virus or the ability to utilize mannose as a carbon source, or both resistance to Beet Necrotic Yellow Vein Virus and the ability to utilize mannose as a carbon source. In one embodiment of this aspect, the transgenic genotype conferring resistance to Beet Necrotic Yellow Vein Virus and the ability to utilize mannose as a carbon comprises a pmi coding sequence. According to one aspect of the invention, Beet Necrotic Yellow Vein Virus resistant sugar beet plants and seeds comprising one or more of the nucleic acid molecules of the invention are provided. One example of a Beet Necrotic Yellow Vein Virus resistant sugar beet plant is the sugar beet for which seed comprising the nucleic acid molecules of the invention have been deposited on Dec. 11, 2008 at NCIMB and assigned the Accession No. 41601. The invention is further directed to plants derived from the Beet Necrotic Yellow Vein Virus resistant sugar beet plant of the present invention for which seed have been deposited at NCIMB under Accession No. 41601. A further aspect is directed to the seeds deposited at NCIMB under Accession No. 41601 as well as to a transgenic Beet Necrotic Yellow Vein Virus resistant sugar beet plant produced, derived or obtained from these seeds.

In another aspect, the present invention provides a biological sample derived from a GM RZ13 sugar beet plant, tissue, or seed, wherein the sample comprises a nucleotide sequence which is or is complementary to a sequence that is unique to event GM RZ13, and wherein the sequence is detectable in the sample using a nucleic acid amplification or nucleic acid hybridization method. In one embodiment of this aspect, the nucleotide sequence is or is complementary to SEQ ID NO: 1, SEQ ID NO: 2, SEQ ID NO: 7 or SEQ ID NO: 8.

In another aspect, the present invention provides an extract derived from a GM RZ13 sugar beet plant, tissue, or seed, wherein the sample comprises a nucleotide sequence which is or is complementary to a sequence that is unique to event GM RZ13, and wherein the sequence is detectable in the sample using a nucleic acid amplification or nucleic acid hybridization method. In one embodiment of this aspect, the nucleotide sequence is or is complementary to SEQ ID NO: 1, SEQ ID NO: 2, SEQ ID NO: 7 or SEQ ID NO: 8. In a preferred embodiment, the nucleic acid amplification or nucleic acid hybridization method usable to detect the nucleotide sequence which is or is complementary to a sequence that is unique to event GM RZ13 in the biological sample of the present invention or the extract of the present invention is the method of detecting the presence of a nucleic acid molecule that is unique to event GM RZ13 of the present invention and as described above.

In another aspect, the present invention provides a method for producing a sugar beet plant resistant to at least Beet Necrotic Yellow Vein Virus comprising (a) sexually crossing a first parent sugar beet plant with a second parent sugar beet plant, wherein said first or second parent sugar beet plant comprises sugar beet event GM RZ13 DNA, thereby producing a plurality of first generation progeny plants; (b) selecting a first generation progeny plant that is resistant to at least Beet Necrotic Yellow Vein Virus; (c) selfing the first generation progeny plant, thereby producing a plurality of second generation progeny plants; and (d) selecting from the second generation progeny plants, a plant that is at least resistant to Beet Necrotic Yellow Vein Virus; wherein the second generation progeny plants comprise a nucleotide sequence that is or is complementary to a nucleotide sequence selected from the group consisting of SEQ ID NO: 1 and SEQ ID NO: 7. In a preferred embodiment of the method for producing a sugar beet plant resistant to at least Beet Necrotic Yellow Vein Virus, said first or second parent sugar beet plant provided in step a) comprising sugar beet event GM RZ13 DNA is the Beet Necrotic Yellow Vein Virus resistant sugar beet plant of the present invention claims and as described hereinabove, or a plant derived from the seeds of the present invention claims and as described hereinabove.

In another aspect, the present invention provides a method for producing Beet Necrotic Yellow Vein Virus resistant sugar beet hybrid seed. Such methods comprise: (a) providing a Beet Necrotic Yellow Vein Virus resistant sugar beet line as a first parent line, (b) providing a second sugar beet line having a different genotype as a second parent line; wherein one of the parent lines of step a) or step b) is a male sterile CMS line and wherein the other parent line is male fertile, and (c) allowing the plants of the male fertile parent line to pollinate the flowers of the male sterile parent line, let the seed develop, and harvest the hybrid seed, wherein the harvested hybrid seeds are seeds of a Beet Necrotic Yellow Vein Virus resistant sugar beet hybrid plant. In an embodiment of this aspect, the male sterile CMS sugar beet parental line provided in step a) or b) is an inbred sugar beet line comprising a nucleotide sequence of the present invention that is unique to event GM RZ13. In a further embodiment of this aspect, the second parental line is selected from the group consisting of (a) an inbred sugar beet plant line resistant to at least Beet Necrotic Yellow Vein Virus having a different genotype but comprising one or more or all nucleotide sequence of the present invention; (b) an inbred sugar beet plant line resistant or tolerant to at least Beet Necrotic Yellow Vein Virus which originates from a naturally occurring source selected from the group comprising the Holly source, WB41, WB42, WB151, WB169, C28, C48, C50, or Rizor or crosses thereof; and (c) an inbred sugar beet plant line having no resistance to the Beet Necrotic Yellow Vein Virus.

Another preferred embodiment of the present invention relates to hybrid seed of a Beet Necrotic Yellow Vein Virus resistant sugar beet plant. In one aspect of the present invention said hybrid seed is produced by the method for producing Beet Necrotic Yellow Vein Virus resistant sugar beet hybrid seed of the present invention. In yet another aspect of the present invention a Beet Necrotic Yellow Vein Virus resistant sugar beet plant is provided that is produced by growing the hybrid seed of the present invention. Preferably, this hybrid plant comprises sugar beet event GM RZ13 DNA. A further preferred embodiment of the present invention relates to a part of said Beet Necrotic Yellow Vein Virus resistant sugar beet plant hybrid plant of the present invention. Preferably said part is selected from the group comprising seeds, microspores, protoplasts, cells, ovules, pollen, vegetative parts, cotyledons, zygotes.

In another aspect, the present invention provides the use of a Beet Necrotic Yellow Vein Virus resistant sugar beet plant or cells or tissues thereof, a biological sample or an extract of the present invention in a method selected from the group comprising of methods of sugar production, methods of aerobic fermentation and methods of anaerobic fermentation. Preferably, said use is the use of a Beet Necrotic Yellow Vein Virus resistant sugar beet plant or cells or tissues thereof, a biological sample or an extract of the present invention in a method of producing sugar.

Further aspects of the present invention are directed to a method of producing sugar, wherein a Beet Necrotic Yellow Vein Virus resistant sugar beet plant, or cells or tissues thereof, a biological sample or an extract of the present invention is processed to produce sugar. Further, sugar is provided by the present invention that is produced by the method of producing sugar of the present invention.

A final aspect relates to a method for producing one or more biofuel(s) selected from the group comprising ethanol, butanol, biogas and/or biodiesel, wherein a Beet Necrotic Yellow Vein Virus resistant sugar beet plant, or cells or tissues thereof, a biological sample or an extract of the present invention is processed to produce one or more biofuel(s) selected from the group comprising ethanol, butanol, biogas and/or biodiesel. Further, biofuel(s) selected from the group comprising ethanol, butanol, biogas and/or biodiesel is/are provided which is/are produced by the method for producing one or more biofuel(s) of the present invention.

The foregoing and other aspects of the invention will become more apparent from the following detailed description.

### DEFINITIONS

The following definitions and methods are provided to better define the present invention and to guide those of ordinary skill in the art in the practice of the present invention. Unless otherwise noted, terms used herein are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag: New York, 1994. The nomenclature for DNA bases and amino acids as set forth in 37 C.F.R. §1.822 is used herein.

As used in this specification and the appended claims, the singular forms “a”, “an”, and “the” include plural referents unless the context clearly dictates otherwise. Thus, for example, reference to “a plant” includes one or more plants, and reference to “a cell” includes mixtures of cells, tissues, and the like.

The terms “Beet Necrotic Yellow Vein Virus” or “BNYVV”, as used herein, refer to the causal agent of rhizomania. The virus belongs to the genus Benyvirus and is transmitted by a soilborne protoctist (Polymyxa betae).

The term “rhizomania”, as used herein, refers to a one of the most important sugar beet diseases worldwide caused by infection with the Beet Necrotic Yellow Vein Virus (BNYVV). Symptoms are yellowing of the plants, stunting, small taproots and an increased number of fibrous roots (also called “root madness”). The vascular tissues of the taproot show a light brown discoloration. If, in rare occasions, the virus spreads systematically to leaves, formation of necrotic vein yellowing is caused.

A “coding sequence” is a nucleic acid sequence that is transcribed into RNA such as mRNA, rRNA, tRNA, snRNA, sense RNA or antisense RNA. Preferably the RNA is then translated in an organism to produce a protein. It may constitute an “uninterrupted coding sequence”, i.e., lacking an intron, such as in a cDNA, or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

As used herein, the term “sugar beet” refers to all species and subspecies within the genus Beta as well as all kinds of cultivated beets of Beta vulgaris any stage of development. Cultivated beets have been separated into four groups: leaf beet, garden beet, fodder beet and sugar beet. “Sugar beet” refers also to all cultivated beets including those grown for other purposes than the production of sugar, such as ethanol, plastics or other industrial products. In particular, “Sugar beet” refers to fodder beet and sugar beet, but especially to sugar beet. The term “sugar beet” also includes sugar beet plants adapted for growth in tropical or subtropical regions.

The term “cultivated” with respect to the sugar beet plants means any sugar beet plant that are commercially grown for their production. The term “cultivated sugar beet plant” includes those plants which has been brought into cultivation and have been selectively bred for growing purposes. Cultivated sugar beet plants exclude those wild-type species which comprise the trait of the present invention as a natural trait and/or part of their natural genetics.

A “sugar beet plant cell” is a structural and physiological unit of a sugar beet plant comprising a protoplast and a cell wall. The sugar beet plant cell may be in the form of an isolated single cell or a cultured cell, or as a part of higher organized unit such as, for example, plant tissue, a plant organ, or a whole plant.

“Sugar beet plant material” refers to leaves, stems, roots, flowers or flower parts, fruits, pollen, egg cells, anthers, ovaries, zygotes, seeds, cuttings, cell or tissue cultures, or any other part or product of a sugar beet plant. This also includes callus or callus tissue as well as extracts (such as extracts from taproots) or samples. Generally, the term “sugar beet plant material” refers to a relatively unprocessed plant material, having intact plant cells.

A “sugar beet plant organ” is a distinct and visibly structured and differentiated part of a sugar beet plant, such as a root, stem, leaf, flower bud, or embryo.

“Sugar beet plant tissue”, as used herein, means a group of sugar beet plant cells organized into a structural and functional unit. Any tissue of a sugar beet plant in planta or in culture is included. This term includes, but is not limited to, whole plants, plant organs, plant seeds, tissue culture and any groups of sugar beet plant cells organized into structural and/or functional units. The use of this term in conjunction with, or in the absence of, any specific type of sugar beet plant tissue as listed above or otherwise embraced by this definition is not intended to be exclusive of any other type of sugar beet plant tissue.

The term “expression”, when used in reference to a nucleic acid sequence, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g., if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In addition, expression refers to the transcription and stable accumulation of sense (mRNA) or functional RNA. Expression may also refer to the production of protein.

“Detection kit”, as used herein, refers to a kit used to detect the presence or absence of DNA from GM RZ13 plants in a sample. The detection kit comprises nucleic acid probes and/or primers of the present invention, which hybridize specifically under high stringency conditions to a target DNA sequence, and other materials necessary to enable nucleic acid hybridization or amplification methods.

As used herein the term transgenic “event” refers to a recombinant sugar beet plant produced by transformation and regeneration of a sugar beet plant cell or tissue with heterologous DNA, for example, with an expression cassette that includes a gene of interest. The term “event” refers to the original transformant and/or progeny of the transformant that include the heterologous DNA. The term “event” also refers to progeny produced by a sexual outcross between the transformant and another sugar beet line. Even after repeated backcrossing to a recurrent parent, the inserted DNA and the flanking DNA from the transformed parent is present in the progeny of the cross at the same chromosomal location. The term “event” also refers to DNA from the original transformant comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA. Normally, transformation of plant tissue produces multiple events, each of which represent insertion of a DNA construct into a different location in the genome of a plant cell. Based on the expression of the transgene or other desirable characteristics, a particular event is selected. The terms “event GM RZ13”, “GM RZ13” or “GM RZ13 event” may be used interchangeably for the sugar beet event GM RZ13 of the present invention. The sugar beet event GM RZ13 is also known as event SBVR111.

As used herein, the term “unique” means distinctively characteristic of event GM RZ13. Therefore, nucleic acids unique to event GM RZ13 are not found in other non-GM RZ13 sugar beet plants.

A GM RZ13 sugar beet plant resistant to Beet Necrotic Yellow Vein Virus can be bred by first sexually crossing a first parental sugar beet plant consisting of a sugar beet plant grown from a transgenic GM RZ13 sugar beet plant, such as a GM RZ13 sugar beet plant grown from the seed deposited at the NCIMB under accession No. 41601, and progeny thereof derived from transformation with the expression cassettes of the embodiments of the present invention that confers resistance to Beet Necrotic Yellow Vein Virus, and a second parental sugar beet plant that lacks resistance to Beet Necrotic Yellow Vein Virus or only shows tolerance towards or only partial resistance against the virus, thereby producing a plurality of first progeny plants; and then selecting a first progeny plant that is resistant to Beet Necrotic Yellow Vein Virus; and selfing the first progeny plant, thereby producing a plurality of second progeny plants; and then selecting from the second progeny plants a plant resistant to Beet Necrotic Yellow Vein Virus. These steps can further include the back-crossing of the first Beet Necrotic Yellow Vein Virus resistant progeny plant or the second Beet Necrotic Yellow Vein Virus resistant progeny plant to the second parental sugar beet plant or a third parental sugar beet plant, thereby producing a sugar beet plant that is resistant to Beet Necrotic Yellow Vein Virus. Plants which are tolerant against the Beet Necrotic Yellow Vein Virus are those plants in which the multiplication rate of the pathogen is high, whereas the development of plant is not restricted. Partial resistance is present in sugar beet plants which get infected, but in which the virus multiplication is lower than in a susceptible genotype.

“Expression cassette”, as used herein, means a nucleic acid molecule capable of directing expression of a particular nucleotide sequence in an appropriate host cell, comprising a promoter operably linked to the nucleotide sequence or sequences of interest which is/are operably linked to termination signals. It also typically comprises sequences required for proper translation of the nucleotide sequence(s). The expression cassette may also comprise sequences not necessary in the direct expression of the nucleotide sequence(s) of interest but which are present due to convenient restriction sites for removal of the cassette from an expression vector. The expression cassette comprising the nucleotide sequence(s) of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation process known in the art. The expression of the nucleotide sequence(s) in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development. An expression cassette, or fragment thereof, can also be referred to as “inserted sequence” or “insertion sequence” when transformed into a plant.

A “gene” is a defined region that is located within a genome and that, besides the aforementioned coding sequence, may comprise other, primarily regulatory, nucleic acid sequences responsible for the control of the expression, that is to say the transcription and translation, of the coding portion. A gene may also comprise other 5′ and 3′ untranslated sequences and termination sequences. Further elements that may be present are, for example, introns.

“Gene of interest” refers to any gene which, when transferred to a plant, confers upon the plant a desired characteristic or phenotype, such as, for example, antibiotic resistance, virus resistance, insect resistance, disease resistance, or resistance to other pests, herbicide tolerance, improved nutritional value, improved performance in an industrial process or altered reproductive capability.

“Genotype,” as used herein, is the genetic material inherited from parent sugar beet plants not all of which is necessarily expressed in the descendant sugar beet plants. The GM RZ13 genotype refers to the heterologous genetic material transformed into the genome of a plant as well as the genetic material flanking the inserted sequence.

A “heterologous” nucleic acid sequence is a nucleic acid sequence not naturally associated with a host cell into which it is introduced, including non-naturally occurring multiple copies of a naturally occurring nucleic acid sequence. The term “heterologous” when used in reference to a gene or nucleic acid refers to a gene encoding a factor that is not in its natural environment (i.e., has been altered by the hand of man). For example, a heterologous gene may include a gene from one species introduced into another species. A heterologous gene may also include a gene native to an organism that has been altered in some way (e.g., mutated, added in multiple copies, linked to a non-native promoter or enhancer sequence, etc.). Heterologous genes further may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). In one aspect of the invention, heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed).

A “homologous” nucleic acid sequence is a nucleic acid sequence naturally associated with a host cell into which it is introduced.

The term “polynucleotide”, as used herein, refers to a polymer of DNA or RNA. The term “nucleic acid” refers to deoxyribonucleotides or ribonucleotides and polymers thereof which can be single- or double-stranded, optionally containing synthetic, non-natural or altered nucleotide bases capable of incorporation into DNA or RNA polymers, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. The term “nucleotide sequence” or “nucleic acid sequence” refers to a polymer of DNA or RNA which can be single or double-stranded, optionally containing synthetic, non-natural or altered nucleotide bases capable of incorporation into DNA or RNA polymers. The terms “nucleic acid” or “nucleic acid sequence” may also be used interchangeably with gene, cDNA, DNA and RNA encoded by a gene.

The term “isolated”, when used in the context of the nucleic acid molecules of the present invention, refers to a nucleic acid sequence that is identified within and isolated/separated from its chromosomal nucleic acid sequence context within the respective source organism. An isolated nucleic acid is not a nucleic acid as it occurs in its natural context, if it indeed has a naturally occurring counterpart. In contrast, non-isolated nucleic acids are nucleic acids such as DNA and RNA, which are found in the state they exist in nature. For example, a given DNA sequence (e.g., a gene) is found on the host cell chromosome in proximity to neighboring genes. The isolated nucleic acid sequence may be present in single-stranded or double-stranded form. Alternatively, it may contain both the sense and anti-sense strands (i.e., the nucleic acid sequence may be double-stranded). If claimed in the context of a plant genome, the nucleic acid molecule of the invention is distinguished over naturally occurring counterparts by the insertion side in the genome and the flanking sequences at the insertion site. In a preferred embodiment, the nucleic acid molecules of the present invention are understood to be isolated.

The terms “protein,” “peptide” and “polypeptide” are used interchangeably herein.

“Operably-linked” refers to the association of nucleic acid sequences on a single nucleic acid fragment so that the function of one affects the function of the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

“Primers”, as used herein, are isolated nucleic acids that are capable of becoming annealed to a complimentary target DNA strand by nucleic acid hybridization to form a hybrid between the primer and the target DNA strand, and then extended along the target DNA strand by a polymerase, such as DNA polymerase. Primer pairs or sets can be used for amplification of a nucleic acid molecule, for example, by the polymerase chain reaction (PCR) or other conventional nucleic-acid amplification methods. A “PCR primer” is understood within the scope of the invention to refer to short fragments of isolated single-stranded DNA used in the PCR amplification of specific regions of DNA.

“PCR” or “Polymerase chain reaction” is understood within the scope of the invention to refer to a method of producing relatively large amounts of specific regions of DNA, thereby making possible various analyses that are based on those regions.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

A “probe” is an isolated nucleic acid to which is attached a conventional detectable label or reporter molecule, such as a radioactive isotope, ligand, chemiluminescent agent, fluorescent label, or enzyme. Such a probe is complimentary to a strand of a target nucleic acid, in the case of the present invention, to a strand of genomic DNA from sugar beet event GM RZ13. The genomic DNA of GM RZ13 can be from a sugar beet plant or from a sample that includes DNA from the event. Probes according to the present invention include not only deoxyribonucleic or ribonucleic acids but also polyamides and other probe materials that bind specifically to a target DNA sequence and can be used to detect the presence of that target DNA sequence.

Primers and probes are generally between 10 and 15 nucleotides or more in length. Primers and probes can also be at least 20 nucleotides or more in length, or at least 25 nucleotides or more, or at least 30 nucleotides or more in length. Such primers and probes hybridize specifically to a target sequence under high stringency hybridization conditions. Primers and probes according to the present invention may have complete sequence complementarity with the target sequence, although probes differing from the target sequence and which retain the ability to hybridize to target sequences may be designed by conventional methods. It is to be understood that the length of the primers and probes of the present invention can be any numerical value between the values specified herein. Thus, primers and probes being generally between 10 and 15 nucleotides or more in length encompass primer and probes having a length of 10, 11, 12, 13, 14, or 15 nucleotides, whereas the expression “at least 20 nucleotides” further includes primer and probes having a length of 16, 17, 18, 19, or nucleotides. The same applies to the expressions “at least 25 nucleotides or more” and “at least 30 nucleotides or more in length”.

“Gene silencing” refers to homology-dependent suppression of viral genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes. Gene silencing includes virus-induced gene silencing.

“RNA interference” (RNAi) refers to the process of sequence-specific post-transcriptional gene silencing in plants and animals mediated by short interfering RNAs (siRNAs). Various terms such as siRNA, target RNA molecule, dicer or ribonuclease III enzyme are concepts known to those skilled in the art and full descriptions of these terms and other concepts pertinent to RNAi can be found in the literature. For reference, several terms pertinent to RNAi are defined below. However, it is understood that any particular hypothesis describing the mechanisms of RNAi are not necessary to practice the present invention.

“dsRNA” or “double-stranded RNA” is RNA with two complementary strands, which directs the sequence-specific degradation of mRNA through a process known as RNA interference (RNAi). dsRNA is cut into siRNAs interfering with the expression of a specific gene.

“Inverted repeat” refers to a nucleotide sequence found at two sites on the same nucleic acid sequence, but in opposite orientation.

The term “siRNAs” refers to short interfering RNAs. In some embodiments, siRNAs comprise a duplex, or double-stranded region, of about 21-23 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of a siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such RNA target molecules include unprocessed mRNA, ribosomal RNA, and viral RNA genomes. It is not necessary that there is 100% homology between the target RNA molecule and the dsRNA over the whole length of the dsRNA, but the hairpins of the dsRNA should comprise stretches of at least 21 nucleotides, preferably of at least 23 nucleotides, more preferred of at least 50 nucleotides, even more preferred of at least 500 nucleotides, most preferred of at least 700 nucleotides, and up to 1000 nucleotides having at least 95%, preferred 100% homology between the target RNA molecule.

As used herein, gene or trait “stacking” is combining desired traits into one transgenic line. Plant breeders stack transgenic traits by making crosses between parents that each have a desired trait and then identifying offspring that have both of these desired traits (so-called breeding stacks). Another way to stack genes is by transferring two or more genes into the cell nucleus of a plant at the same time during transformation. Another way to stack genes is by re-transforming a transgenic plant with another gene of interest. For example, gene stacking can be used to combine two different insect resistance traits, an insect resistance trait and a disease resistance trait, or a disease resistance trait with a herbicide resistance trait (such as, for example, glyphosate resistance). The use of a selectable marker in addition to a gene of interest would also be considered gene stacking. Traits of interest for stacking are GM traits and non-GM traits GM traits of interest include, for example, herbicide resistance, insect resistance, disease resistance, transgenic plants having a phenotype of delayed or inhibited bolting, transgenic plants with changed and/or enhanced carbohydrate composition. Non-GM traits of interest include, for example, disease resistance or resistance against BNYVV from conventional sources (like Holly, WB41, WB42, WB151, WB169, C28, C48, C50, or Rizor, or crosses thereof) or viruses other than BNYVV, or tolerance to pests like, for example, beet cyst nematodes, root aphids, root knot nematodes, or tolerance to fungal pests like, for example, Cercospora, Aphanomyces, Rhizoctonia, Fusarium, Ramularia, Erysipe, Peronospora, Erwinia, Sclerotium, Verticillium, Phoma, or Rust, or tolerance to viruses like, for example, Beet Curly Top Virus, Beet Yellow Virus, Beet Mild Yellow Virus, Beet Western Yellow Virus.

“Stringent hybridization conditions” and “stringent hybridization wash conditions” in the context of nucleic acid hybridization experiments, such as Southern and Northern hybridizations, are sequence dependent, and are different under different environmental parameters. Longer sequences hybridize specifically at higher temperatures. An extensive guide to the hybridization of nucleic acids is found in Tijssen (1993). High stringency hybridization conditions are described, for example, in Sambrook et al. An example of high stringency hybridization conditions for hybridization of complementary nucleic acids which have more than 100 complementary residues on a filter in a Southern or northern blot is 50% formamide with 1 mg of heparin at 42° C., with the hybridization being carried out overnight. An example of very high stringency wash conditions is 0.15M NaCl at 72° C. for about 15 minutes. An example of high stringency wash conditions is a 0.2×SSC wash at 65° C. for 15 minutes (see, Sambrook) for a description of SSC buffer).

“Transformation” is a process for introducing heterologous nucleic acid into a host cell or organism. In particular, “transformation” means the stable integration of a DNA molecule into the genome of an organism of interest.

“Transformed/transgenic/recombinant” refers to a plant organism into which a heterologous nucleic acid molecule has been introduced. The nucleic acid molecule can be stably integrated into the genome of the host plant or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type plant organism, which does not contain the heterologous nucleic acid molecule. As used herein, “transgenic” refers to a plant, plant cell, or multitude of structured or unstructured plant cells having integrated, via well known techniques of genetic manipulation and gene insertion, a sequence of nucleic acid representing a gene of interest into the plant genome, and typically into a chromosome of a cell nucleus, mitochondria or other organelle containing chromosomes, at a locus different to, or in a number of copies greater than, that normally present in the native plant or plant cell. Transgenic plants result from the manipulation and insertion of such nucleic acid sequences, as opposed to naturally occurring mutations, to produce a non-naturally occurring plant or a plant with a non-naturally occurring genotype. Techniques for transformation of plants and plant cells are well known in the art and may comprise for example electroporation, microinjection, Agrobacterium-mediated transformation, and ballistic transformation.

A “transgenic plant” is a plant having one or more plant cells that contain an expression vector.

The term “sugar” refers to fermentable monosaccharides disaccharides, and trisaccharides, particularly to mono- and disaccharides. Thus, in the present invention, sugars include, but are not limited to, sucrose, fructose, glucose, galactose, maltose, lactose, and mannose.

The term “biofuel”, as used herein, refers to a fuel that is derived from biomass, i.e., a living or recently living biological organism, such as a plant or an animal waste. Biofuels include, but are not limited to, biodiesel, biohydrogen, biogas, biomass-derived dimethylfuran (DMF), and the like. In particular, the term “biofuel” can be used to refer to plant-derived alcohols, such as ethanol, methanol, propanol, or butanol, which can be denatured, if desired prior to use. The term “biofuel” can also be used to refer to fuel mixtures comprising plant-derived fuels, such as alcohol/gasoline mixtures (i.e., gasohols). Gasohols can comprise any desired percentage of plant-derived alcohol (i.e., about 10%, 15%, 20%, 25%, 30%, 35%, 40%, 45%, 50%, 55%, 60%, 65%, 70%, 75%, 80%, 85%, 90%, or 95% plant-derived alcohol). For example, one useful biofuel-based mixture is E85, which comprises 85% ethanol and 15% gasoline. The term “biofuel” refers to any biofuel produced by aerobic or anaerobic fermentation of plant material.

“Fermentation” as used herein refers to the process of transforming an organic molecule into another molecule using a microorganism. If not indicated otherwise the term “fermentation” includes anaerobic and aerobic fermentation. Methods of aerobic and/or anaerobic fermentation are known to the person skilled in the art.

### BRIEF DESCRIPTION OF THE FIGURES AND SEQ IDs

FIG. 1 depicts a map of binary vector pSYN15965 (previously known as pHiNK188) containing the RNAi construct (including sequence obtained from BNYVV RNA1) used for transforming sugar beet.

FIG. 2 depicts the results of a Northern blot analysis of the accumulation of mRNA (FIG. 2, upper panel) and siRNA (FIG. 2, lower panel) resulting from the transgenic expression of the BNYVV replicase gene-derived inverted repeat. Roots were harvested 0, 7, 14, 21 or 28 days after transplantation into infested soil with B-type BNYVV or sterile sand. Figure legend: a—transgenic plant grown in sterile sand; b—transgenic plant grown in infested soil; c—non-transgenic plant grown in infested soil; d—non transgenic plant grown in sterile sand; l—leaf of Nicotiana benthamiana plant agro-infiltrated with the binary vector pHiNK188 used for the transformation of the sugar beet. 5S rRNA (at the bottom) was used as control for equal loading of RNA.

FIG. 3 depicts the results of an ELISA assay of the titer of BNYVV in brei samples from sugar beets grown in a field trial in the USA in 2009 (see example 7). The mean value of the BNYVV content per group [ng BNYV/ml sap] is: 88 with a standard deviation of 39 for “Holly” (16 plants tested), 125 with a standard deviation of 18 for “Holly+C48” (3 plants tested), and 5 with a standard deviation of 5 for “Holly+GMRZ” (9 plants tested), respectively. The vertical lines represent the mean value.

SEQ ID NO: 1 is the 3′ junction sequence of the GM RZ13 event.

SEQ ID NO: 2 is a sequence of 807 nucleotides spanning 460 nucleotides of the 3′ end of the RZ insert (nucleotides 1-460; right border) and 347 nucleotides of sugar beet genomic DNA flanking the insert (nucleotides 461-807) in the GM RZ13 event.

SEQ ID NO: 3 is the sequence of sugar beet genomic DNA (347 nucleotides) flanking the 3′ end of the RZ insert in the GM RZ13 event.

SEQ ID NO: 4 is the sequence of primer FE1005.

SEQ ID NO: 5 is the sequence of primer FE1006.

SEQ ID NO: 6 is the sequence of primer FE1007.

SEQ ID NO: 7 is the 5′ junction sequence of the GM RZ13 event.

SEQ ID NO: 8 is a sequence of 484 nucleotides spanning 247 nucleotides of sugar beet genomic DNA flanking the 5′ end of the RZ insert (nucleotides 238-484) and 237 nucleotides of the 5′ end of the RZ insert (nucleotides 1-237; left border) in the GM RZ13 event.

SEQ ID NO: 9 is the sequence of sugar beet genomic DNA (237 nucleotides) flanking the 5′ end of the RZ insert in the GM RZ13 event.

SEQ ID NO: 10 is the sequence of primer ESPCR0008.

SEQ ID NO: 11 is the sequence of primer FE0902.

SEQ ID NO: 12 is the sequence of primer FE02226.

SEQ ID NO: 13 is the sequence of primer FE02216.

SEQ ID NO: 14 is the sequence of primer FE02236.

SEQ ID NO: 15 is the sequence of primer FE02237.

SEQ ID NO: 16 is the sequence of primer FE02238.

SEQ ID NO: 17 is the sequence of primer FE0622.

SEQ ID NO: 18 is the sequence of primer FlkSeq0008.

SEQ ID NO: 19 is the sequence of primer FE0820.

SEQ ID NO: 20 is the sequence of primer FlkSeq0010.

SEQ ID NO: 21 is the sequence of primer FE0885.

SEQ ID NO: 22 is the sequence of primer FE0927.

SEQ ID NO: 23 is the sequence of primer FE02201.

SEQ ID NO: 24 is the sequence of primer FE02202.

SEQ ID NO: 25 is the sequence of primer FE06202.

SEQ ID NO: 26 is the sequence of primer FE06311.

SEQ ID NO: 27 is the sequence of probe FE06312.

SEQ ID NO: 28 is the sequence of primer HiNK285.

SEQ ID NO: 29 is the sequence of primer HiNK283.

SEQ ID NO: 30 is the sequence of primer HiNK284.

### DETAILED DESCRIPTION

This invention relates to a genetically improved line of sugar beet that comprises an inverted repeat comprising a fragment from the BNYVV replicase gene, and produces a phosphomannose isomerase enzyme (PMI) that allows the plant to utilize mannose as a carbon source. The invention is particularly drawn to a transgenic sugar beet event designated GM RZ13 (or SBVR111, a designation that is used interchangeably with the designation GM RZ13) comprising a novel genotype, as well as to compositions and methods for detecting nucleic acids from this event in a biological sample. The invention is further drawn to sugar beet plants comprising the GM RZ13 genotype, to transgenic seed from the sugar beet plants, and to methods for producing a sugar beet plant comprising the GM RZ13 genotype by crossing a sugar beet inbred comprising the GM RZ13 genotype with itself or another sugar beet line, the production of hybrids comprising the GM RZ13 genotype and the use of the BNYVV resistant plants of the present invention for the production of sugar or in aerobic or anaerobic fermentation, e.g. for the production of biofuel(s).

In one embodiment, the present invention encompasses a nucleic acid molecule, particularly an isolated nucleic acid molecule, comprising a nucleotide sequence that is unique to event GM RZ13.

In a preferred embodiment, the nucleic acid molecule comprising a nucleotide sequence that is unique to event GM RZ13 is a nucleic acid molecule, particularly an isolated nucleic acid molecule, that links a heterologous DNA molecule inserted into the sugar beet plant genome of event GM RZ13 to the sugar beet plant genome DNA in event GM RZ13 and comprises at least 10 or more (for example 15, 20, 25, 30, 35, 40, 45, or 50) contiguous nucleotides of the heterologous DNA molecule and at least 10 or more (for example 15, 20, 25, 30, 35, 40, 45, or 50) contiguous nucleotides of the sugar beet plant genome DNA flanking the point of insertion of the heterologous DNA molecule. In further preferred embodiments, the nucleic acid molecule comprising a nucleotide sequence that is unique to event GM RZ13 is a nucleic acid molecule, particularly an isolated nucleic acid molecule, that links a heterologous DNA molecule inserted into the sugar beet plant genome of event GM RZ13 to the sugar beet plant genome DNA in event GM RZ13 and comprises at least 10, preferably at least 20, and more preferably at least 50 contiguous nucleotides of the heterologous DNA molecule and at least 10, preferably at least 20, and more preferably at least 50 contiguous nucleotides of the genome DNA flanking the point of insertion of the heterologous DNA molecule. Also included are nucleotide sequences that comprise 10 or more nucleotides of contiguous insert sequence from event GM RZ13 and at least one nucleotide of flanking DNA from event GM RZ13 adjacent to the insert sequence. Such nucleotide sequences are unique to and diagnostic for event GM RZ13. Nucleic acid amplification of genomic DNA from sugar beet event GM RZ13 produces an amplicon comprising such unique sequences and is diagnostic for event GM RZ13. In one aspect of this embodiment, the nucleotide sequence that is unique to event GM RZ13 is selected from the group consisting of SEQ ID NO: 1 (the junction sequence of the GM RZ13 event), SEQ ID NO: 2 (the sequence spanning 460 nucleotides of the 3′ end of the RZ insert and 347 nucleotides of sugar beet genomic DNA flanking the insert in the GM RZ13 event.), SEQ ID NO: 7 (the 5′ junction sequence of the GM RZ13 event), SEQ ID NO: 8 (the sequence spanning 247 nucleotides of sugar beet genomic DNA flanking the 5′ end of the RZ insert and 237 nucleotides of the 5′ end of the RZ insert in the GM RZ13 event), and the complements thereof.

In another embodiment, the invention encompasses a nucleic acid molecule, particularly an isolated nucleic acid molecule, comprising a nucleotide sequence which comprises at least one junction sequence of event GM RZ13, wherein a junction sequence spans the junction between a heterologous expression cassette inserted into the sugar beet genome and DNA from the sugar beet genome flanking the insertion site and is diagnostic for the event. In one aspect of this embodiment, the junction sequence is selected from the group consisting of SEQ ID NO: 1, SEQ ID NO: 7, and the complements thereof. The junction sequences span the junction between the heterologous expression cassette inserted into the sugar beet genome and DNA from the sugar beet genome flanking the insertion site. As there is only one heterologous expression cassette inserted into the sugar beet genome giving rise to event GM RZ13, both the junction sequence comprising the 5′ end of the heterologous expression cassette linked to flanking genomic sequence and the junction sequence comprising the 3′ end of the heterologous expression cassette linked to flanking genomic sequence, respectively, are unique to event GM RZ13. Due to their unique nature these sequences are diagnostic for the event

According to another aspect, the present invention provides a nucleic acid, particularly an isolated nucleic acid, linking a heterologous DNA molecule to the sugar beet plant genome in sugar beet event GM RZ13 comprising a sequence of from about 11 to about 20 contiguous nucleotides. In one embodiment of this aspect, the nucleic acid is selected from the group consisting of SEQ ID NO: 1, SEQ ID NO: 7, and the complements thereof.

In another embodiment, the invention encompasses a nucleic acid molecule, particularly an isolated nucleic acid molecule, comprising a nucleotide sequence selected from the group consisting of SEQ ID NO: 1, SEQ ID NO: 2, SEQ ID NO: 3 (the sequence of sugar beet genomic DNA flanking the 3′ end of the RZ insert in the GM RZ13 event), SEQ ID NO: 7, SEQ ID NO: 8, SEQ ID NO: 9 (the sequence of sugar beet genomic DNA flanking the 5′ end of the RZ insert in the GM RZ13 event), and the complements thereof. In one aspect of this embodiment, the nucleic acid molecule is comprised in a sugar beet seed deposited at NCIMB under the accession No. 41601.

In one embodiment of the present invention, an amplicon comprising a nucleotide sequence unique to event GM RZ13 is provided. In one aspect of this embodiment, the amplicon of the present invention comprises a nucleotide sequence of the present invention and as described hereinabove, preferably selected from the group consisting of SEQ ID NO: 1, SEQ ID NO: 2, SEQ ID NO: 7, SEQ ID NO: 8, and the complements thereof.

According to another aspect, the present invention provides a pair of polynucleotide primers comprising a first polynucleotide primer and a second polynucleotide primer that function together in the presence of a sugar beet event GM RZ13 DNA template in a sample to produce an amplicon diagnostic for event GM RZ13.

In one embodiment of this aspect, one of said primer sequence is or is complementary to a sugar beet plant genome sequence flanking the point of insertion of a heterologous DNA sequence inserted into the sugar beet plant genome of sugar beet event GM RZ13, and the other polynucleotide primer sequence is or is complementary to the heterologous DNA sequence inserted into the sugar beet plant genome of the sugar beet event GM RZ13.

It is clear to the person skilled in the art that generally one of said primer sequences can be derived from the T-DNA insert inserted into the genome of the sugar beet, whereas the other primer sequence can be derived from the sequence flanking the insert; or that one of the primer sequences can be derived from one of the junction sequences, whereas the other primer sequences can be derived from either the T-DNA insert or the sequence flanking the insert. It has to be noted in this context, that the expression “one of the primer sequences” could mean the first primer sequence used in the PCR reaction (i.e., either a forward or a reverse primer) and “the other polynucleotide primer sequence” could mean the second primer sequence used in the PCR reaction (i.e., either a forward or a reverse primer) (or vice versa).

In preferred embodiments, the first polynucleotide primer comprised in the pair of polynucleotide primers of the present invention is derived from sequence flanking the GM RZ insert, while the second polynucleotide primer is derived from sequence of the insert inserted into the genome of the sugar beet.

In one embodiment of this aspect, one of the primer sequences is chosen from SEQ ID NO: 2 (the sequence spanning 460 nucleotides of the 3′ end of the RZ insert and 347 nucleotides of sugar beet genomic DNA flanking the insert in the GM RZ13 event.), SEQ ID NO:3 (the sequence of sugar beet genomic DNA flanking the 3′ end of the RZ insert in the GM RZ13 event), SEQ ID NO: 8 (the sequence spanning 247 nucleotides of sugar beet genomic DNA flanking the 5′ end of the RZ insert and 237 nucleotides of the 5′ end of the RZ insert in the GM RZ13 event) or SEQ ID NO: 9 (the sequence of sugar beet genomic DNA flanking the 5′ end of the RZ insert in the GM RZ13 event). It has to be noted again, that the expression “one of the primer sequences” could mean the first primer sequence used in the PCR reaction (i.e., either a forward or a reverse primer).

In another embodiment of this aspect, the first polynucleotide primer comprises at least 10 contiguous nucleotides from SEQ ID NO: 3 or from position 461-807 of SEQ ID NO: 2, or comprises at least 10 contiguous nucleotides from SEQ ID NO: 9 or from position 1-237 of SEQ ID NO: 8, and the complements thereof. Accordingly, the first polynucleotide primer comprises sequences derived from sequence flanking the RZ insert inserted into the genomic DNA of event GM RZ13.

In a further preferred embodiment, the first polynucleotide comprised in the primer pair of polynucleotide primers of the present invention comprises a nucleotide sequence selected from the group consisting of SEQ ID NO: 12, SEQ ID NO: 13, SEQ ID NO: 14, SEQ ID NO: 15, SEQ ID NO: 16, SEQ ID NO: 23, SEQ ID NO: 24, SEQ ID NO: 25, and the complements thereof.

In another embodiment of this aspect, the second polynucleotide primer comprises at least 10 contiguous nucleotides from position 1-460 as set forth as SEQ ID NO: 2, or derived from position 238-484 as set forth as SEQ ID NO: 8, or the complements thereof. Accordingly, the second polynucleotide primer comprises sequences derived from the RZ insert inserted into the genomic DNA of event GM RZ13.

In still another embodiment of this aspect, the second polynucleotide primer comprised in the primer pair of polynucleotide primers of the present invention comprises a nucleotide sequence selected from the group consisting of SEQ ID NO: 12, SEQ ID NO: 13, SEQ ID NO: 14, SEQ ID NO: 15, SEQ ID NO: 16, SEQ ID NO: 23, SEQ ID NO: 24, SEQ ID NO: 25, and the complements thereof.

In yet another embodiment of this aspect, the pair of primers of the present invention is selected from the group of primer pairs consisting of: (a) the polynucleotide primer as set forth as SEQ ID NO: 13 as the first polynucleotide primer and the second polynucleotide primer as set forth as SEQ ID NO: 18, and complements thereof; (b) the polynucleotide primer as set forth as SEQ ID NO: 14 as the first polynucleotide primer and the second polynucleotide primer as set forth as either SEQ ID NO: 10 or SEQ ID NO: 18, and complements thereof; (c) the polynucleotide primer as set forth as SEQ ID NO: 15 as the first polynucleotide primer and the second polynucleotide primer as set forth as SEQ ID NO: 18, and complements thereof; (d) the polynucleotide primer as set forth as SEQ ID NO: 16 as the first polynucleotide primer and the second polynucleotide primer as set forth as SEQ ID NO: 18, and complements thereof; (e) the polynucleotide primer as set forth as SEQ ID NO: 12 as the first polynucleotide primer and the second polynucleotide primer as set forth as either SEQ ID NO: 19, SEQ ID NO: 4, SEQ ID NO: 5, or SEQ ID NO: 6, and complements thereof; (f) the polynucleotide primer as set forth as SEQ ID NO: 19 as the first polynucleotide primer and the second polynucleotide primer as set forth as SEQ ID NO: 24, and complements thereof; (g) the polynucleotide primer as set forth as SEQ ID NO: 20 as the first polynucleotide primer and the second polynucleotide primer as set forth as SEQ ID NO: 24, and complements thereof; and (h) the polynucleotide primer as set forth as SEQ ID NO: 25 as the first polynucleotide primer and the second polynucleotide primer as set forth as SEQ ID NO: 26, and complements thereof.

In a further aspect of the present invention, pairs of primers are provided which are selected from the group of primer pairs consisting of: (a) the polynucleotide primer as set forth as SEQ ID NO: 13 as the first polynucleotide primer and the second polynucleotide primer as set forth as either SEQ ID NO: 11 or SEQ ID NO: 17, and complements thereof; (b) the polynucleotide primer as set forth as SEQ ID NO: 12 as the first polynucleotide primer and the second polynucleotide primer as set forth as either SEQ ID NO: 21 or SEQ ID NO: 22, and complements thereof; (c) the polynucleotide primer as set forth as SEQ ID NO: 19 as the first polynucleotide primer and the second polynucleotide primer as set forth as SEQ ID NO: 23, and complements thereof; and (d) the polynucleotide primer as set forth as SEQ ID NO: 20 as the first polynucleotide primer and the second polynucleotide primer as set forth as SEQ ID NO: 23, and complements thereof. The polynucleotide primers set forth as SEQ ID NOs: 11, 17, 21 and 22 are derived from flanking sequences which go beyond the flanking sequences provided herein as SEQ ID NOs: 3 and 9.

Of course, it is well within the skill in the art to obtain additional sequence further out into the genome sequence flanking either end of the inserted heterologous DNA sequences for use as a primer sequence that can be used in such primer pairs for amplifying the sequences that are diagnostic for the GM RZ13 event. For the purposes of this disclosure, the phrase “further out into the genome sequence flanking either end of the inserted heterologous DNA sequences” refers specifically to a sequential movement away from the ends of the inserted heterologous DNA sequences, the points at which the inserted DNA sequences are adjacent to native genomic DNA sequence, and out into the genomic DNA of the particular chromosome into which the heterologous DNA sequences were inserted. Preferably, a primer sequence corresponding to or complementary to a part of the insert sequence should prime the transcriptional extension of a nascent strand of DNA or RNA toward the nearest flanking sequence junction. Consequently, a primer sequence corresponding to or complementary to a part of the genomic flanking sequence should prime the transcriptional extension of a nascent strand of DNA or RNA toward the nearest flanking sequence junction. A primer sequence can be, or can be complementary to, a heterologous DNA sequence inserted into the chromosome of the plant, or a genomic flanking sequence. One skilled in the art would readily recognize the benefit of whether a primer sequence would need to be, or would need to be complementary to, the sequence as set forth within the inserted heterologous DNA sequence depending upon the nature of the product desired to be obtained through the use of the nested set of primers intended for use in amplifying a particular flanking sequence containing the junction between the genomic DNA sequence and the inserted heterologous DNA sequence.

According to another aspect of the invention, a method of detecting the presence of a nucleic acid molecule that is unique to event GM RZ13 in a biological sample is provided. Such methods comprise: (a) contacting the sample comprising DNA with a pair of primers that, when used in a nucleic-acid amplification reaction with genomic DNA from sugar beet event GM RZ13, produces an amplicon that is diagnostic for sugar beet event GM RZ13; (b) performing a nucleic acid amplification reaction, thereby producing the amplicon; and (c) detecting the amplicon. In one embodiment, the pair of primers applied in step a) of said method is a pair of primers of the present invention and as disclosed hereinabove. In another preferred embodiment, one of the primers of the pair of primers applied in step a) of said method is a primer of the present invention and as disclosed hereinabove. In yet another embodiment of this aspect, the amplicon produced and detected in said method comprises a nucleotide sequence selected from the group consisting of SEQ ID NO: 1, SEQ ID NO: 2, SEQ ID NO: 3, SEQ ID NO: 7, SEQ ID NO: 8, SEQ ID NO: 9, and complements thereof.

In a further preferred embodiment of this aspect, said method of detecting the presence of a nucleic acid molecule that is unique to event GM RZ13 in a biological sample is either a gel-based assay or a TaqMan® assay. Other methods of detecting the presence of a nucleic acid molecule that is unique to event GM RZ13 in a biological sample are known to the person skilled in the art. Preferably, the gel-based assay comprises the steps of (i) contacting the sample comprising sugar beet nucleic acids with a pair of primers having the sequence as set forth as SEQ ID NOs: 5 and 12 or a pair of primers having the sequence as set forth as SEQ ID NOs: 13 and 18; (ii) performing a nucleic acid amplification reaction, thereby producing the amplicon; and (iii) detecting the amplicon. A further preferred embodiment is a TaqMan® assay comprising the steps of (i) contacting the sample comprising sugar beet nucleic acids with a pair of primers having the sequence as set forth as SEQ ID NOs: 25 and 26 and a TaqMan® probe having the sequence as set forth as SEQ ID NO: 27; (ii) performing a nucleic acid amplification reaction, thereby producing the amplicon; and (iii) detecting the increase in fluorescence emitted by the reporter dye cleaved from the probe and separated from the quencher dye during the amplification in step (ii). Standard conditions for running a gel-based assay and a TaqMan® assay are known to the person skilled in the art. In both the gel-based assay and the TaqMan® assay the primer pair used in step (a) can be any primer pair of the present invention and as described hereinabove. The TaqMan® probe used in the TaqMan® assay is labeled with a 5′ reporter dye and a 3′ quencher dye will anneal to the sugar beet nucleic acids in the sample. While the probe is intact, the quencher suppresses the fluorescence of the reporter dye. During the nucleic acid amplification reaction in step (b) the Taq DNA polymerase cleaves the probe and thereby displaces it from the sugar beet nucleic acids while the amplicon is produced. During cleavage of the probe the reporter dye is separated from the quencher dye resulting in an increase in fluorescence. The increased fluorescence only occurs if the target sequence is amplified and is complimentary to the probe, thus preventing detection of non-specific amplification.

In another embodiment, the present invention encompasses a method of detecting the presence of a nucleic acid molecule that is unique to event GM RZ13 in a sample comprising sugar beet nucleic acids, wherein the method comprises: (a) contacting the sample comprising sugar beet nucleic acids with a probe that hybridizes under high stringency conditions with genomic DNA from sugar beet event GM RZ13 and does not hybridize under high stringency conditions with DNA from a control sugar beet plant; (b) subjecting the sample and probe to high stringency hybridization conditions; and (c) detecting hybridization of the probe to the DNA. Detection of the amplicon or the probe can be conducted by any means well known in the art including but not limited to fluorescent, chemiluminescent, radiological, immunological, or otherwise. In the case in which hybridization is intended to be used as a means for amplification of a particular sequence to produce an amplicon which is diagnostic for the GM RZ13 sugar beet event, the production and detection by any means well known in the art of the amplicon is intended to be indicative of the intended hybridization to the target sequence where one probe or primer is utilized, or sequences where two or more probes or primers are utilized.

“Highly stringent conditions” or “highly stringent hybridization conditions” include reference to conditions under which a probe will hybridize to its target sequence, to a detectably greater degree than to other sequences. Highly stringent conditions are target-sequence-dependent and will differ depending on the structure of the polynucleotide. By controlling the stringency of the hybridization and/or wash conditions, target sequences can be identified which are 100% complementary to the probe (homologous probing). Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, Part I, Chapter 2 “Overview of principles of hybridization and the strategy of nucleic acid probe assays”, Elsevier: New York; and Current Protocols in Molecular Biology, Chapter 2, Ausubel et al., Eds., Greene Publishing and Wiley-Interscience: New York (1995), and also Sambrook et al. (2001) Molecular Cloning: A Laboratory Manual (5^{th} Ed. Cols Spring Harbor Laboratory, Cold Spring Harbor, N.Y.).

Specificity is typically the function of post-hybridization washes, the critical factors being the ionic strength and temperature of the final wash solution. Generally, high stringency hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, under high stringency conditions a probe will hybridize to its target subsequence, but to no other sequences.

An example of high stringency hybridization conditions for hybridization of complementary nucleic acids which have more than 100 complementary residues on a filter in a Southern or northern blot is 50% formamide with 1 mg of heparin at 42° C., with the hybridization being carried out overnight. An example of very high stringency wash conditions is 0.15M NaCl at 72° C. for about 15 minutes. An example of high stringency wash conditions is a 0.2×SSC wash at 65° C. for 15 minutes (see, Sambrook, infra, for a description of SSC buffer).

For probes of about 10 to 50 nucleotides, high stringency conditions typically involve salt concentrations of less than about 1.0 M Na ion, typically about 0.01 to 1.0 M Na ion concentration (or other salts) at pH 7.0 to 8.3, and the temperature is typically at least about 30° C. High stringency conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under high stringency conditions are still substantially identical if the proteins that they encode are substantially identical. This occurs, e.g., when a copy of a nucleic acid is created using the maximum codon degeneracy permitted by the genetic code.

The following are exemplary sets of hybridization/wash conditions that may be used to hybridize nucleotide sequences that are substantially identical to reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C. The sequences of the present invention may be detected using all the above conditions. For the purposes of defining the invention, the high stringency conditions are used.

It is well within the skill in the art to use different methods for detecting the presence of a nucleic acid molecule that is unique to event GM RZ13 in a sample. Examples of such methods, which are encompassed by the present invention, include, but are not limited to the detection based on RNA or proteins. Examples of such further methods are ELISA, lateral flow strips, and dipsticks. The detection based on RNA may be directed to the detection of siRNAs of the sequence derived from RNA1 of BNYVV included in the insert of GM RZ13. The detection based on protein (using, for example, ELISA, lateral flow sticks, or dipsticks) may be directed to the detection of the PMI protein included into the insert of GM RZ13 or the protein targeted by the RNAi construct in the insert of GM RZ13. Such methods of detecting RNA and proteins are known to the person skilled in the art.

The term “biological sample” is intended to comprise a sample that contains or is suspected of containing a nucleic acid comprising from between five and ten nucleotides either side of the point at which one or the other of the two terminal ends of the inserted heterologous DNA sequence contacts the genomic DNA sequence within the chromosome into which the heterologous DNA sequence was inserted, herein also known as the junction sequences. In addition, the junction sequence comprises as little as two nucleotides: those being the first nucleotide within the flanking genomic DNA adjacent to and covalently linked to the first nucleotide within the inserted heterologous DNA sequence. In one aspect of this embodiment, the amplicon or probe comprises a nucleotide sequence derived from the group consisting of SEQ ID NO: 1, SEQ ID NO: 2, SEQ ID NO: 3, SEQ ID NO: 7, SEQ ID NO: 8, SEQ ID NO: 9, and the complements thereof. “Derived” in this context means, that the amplicon or probe may comprise the complete sequence of one of the sequences set forth as SEQ ID NO: 1, SEQ ID NO: 2, SEQ ID NO: 3, SEQ ID NO: 7, SEQ ID NO: 8, SEQ ID NO: 9, and the complements thereof, or just a fragment or part thereof.

In yet another embodiment, the present invention encompasses a kit for the detection of nucleic acids that are unique to event GM RZ13 in biological sample. The kit comprises at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to event GM RZ13 in the sample. The kit further comprises other materials necessary to enable nucleic acid hybridization or amplification methods. In one aspect of this embodiment, said nucleic acid molecule contained in the kit can be any of the sequences of the present invention and as described hereinabove, but preferably comprises a nucleotide sequence selected from the group consisting of SEQ ID NO: 1, SEQ ID NO: 4, SEQ ID NO: 5, SEQ ID NO: 6, SEQ ID NO: 7, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and the complements thereof. The expression “nucleic acid molecule of sufficient length of contiguous polynucleotides to function as a primer or probe” is meant to refer to both sequences that (1) correspond to the specific nucleotide sequences given above or to sequences that (2) are derived from the specific nucleotide sequences given above.

A variety of detection methods can be used including, but not limited to TAQMAN (Perkin Elmer), thermal amplification, ligase chain reaction, southern hybridization, ELISA methods, and colorimetric and fluorescent detection methods. In particular the present invention provides for kits for detecting the presence of the target sequence, i.e., at least the inverted repeat comprising a fragment from the BNYVV replicase gene sequence or a junction sequence, in a sample containing genomic nucleic acid from GM RZ13. The kit is comprised of at least one polynucleotide capable of binding to the target site or substantially adjacent to the target site and at least one means for detecting the binding of the polynucleotide to the target site. The detecting means can be fluorescent, chemiluminescent, colorimetric, or isotopic and can be coupled at least with immunological methods for detecting the binding. A kit is also envisioned which can detect the presence of the target site in a sample, i.e., at least the inverted repeat comprising a fragment from the BNYVV replicase gene sequence or a junction sequence of GM RZ13, taking advantage of two or more polynucleotide sequences which together are capable of binding to nucleotide sequences adjacent to or within about 100 base pairs, or within about 200 base pairs, or within about 500 base pairs or within about 1000 base pairs of the target sequence and which can be extended toward each other to form an amplicon which contains at least the target site.

The present invention further provides a sugar beet plant comprising the transgenic genotype of the invention, wherein the transgenic genotype confers upon the sugar beet plant resistance to Beet Necrotic Yellow Vein Virus or the ability to utilize mannose as a carbon source, or both resistance to Beet Necrotic Yellow Vein Virus and the ability to utilize mannose as a carbon source. In one embodiment of this aspect, the transgenic genotype conferring resistance to Beet Necrotic Yellow Vein Virus and the ability to utilize mannose as a carbon comprises a pmi coding sequence. According to one aspect of the invention, Beet Necrotic Yellow Vein Virus resistant sugar beet plants and seeds comprising a nucleic acid molecule of the invention comprising a nucleotide sequence that is unique to event GM RZ13 are provided. One example of a Beet Necrotic Yellow Vein Virus resistant sugar beet plants is the sugar beet for which seed comprising the nucleic acid molecules of the invention have been deposited on Dec. 11, 2008 at NCIMB under NCIMB accession No. 41601. The invention is further directed to plants derived from the Beet Necrotic Yellow Vein Virus resistant sugar beet plant of the present invention for which seed have been deposited at NCIMB under Accession No. 41601. Another aspect of the present invention is related to sugar beet seed comprising a nucleic acid molecule of the invention comprising a nucleotide sequence that is unique to event GM RZ13. A further aspect is directed to the seeds deposited at NCIMB under Accession No. 41601 as well as to a transgenic Beet Necrotic Yellow Vein Virus resistant sugar beet plant derived from these seeds. In addition, the present invention is also directed to progeny of the transgenic BNYVV resistant sugar beet plant deposited at the NCIMB under the accession number 41601. “Derived” in the context of plants derived from the Beet Necrotic Yellow Vein Virus resistant sugar beet plant of the present invention or of transgenic Beet Necrotic Yellow Vein Virus resistant sugar beet plant derived from the seeds deposited at NCIMB under Accession No. 41601 means plants produced or obtained from said Beet Necrotic Yellow Vein Virus resistant sugar beet plant or said seeds.

The transgenic sugar beet plant of the present invention is resistant to Beet Necrotic Yellow Vein Virus and is referred to as GM RZ13 (or SBVR111). The transgenic GM RZ13 sugar beet expresses an inverted repeat (RZM) of a part of the RNA-1 gene transcript of the BNYVV (see Example 1 below). This portion of RNA-1 encodes the RNA dependent RNA polymerase (RdRp) or replicase protein. Expression of the RZM, driven by the promoter and intron from the Ubiquitin3 (Ubi3) gene of Arabidopsis thaliana, confers resistance to BNYVV by targeting the replicase RNA transcript of the infecting virus via an RNAi mechanism and thus by interacting with the reproductive system of the virus. This interaction leads to a reduction of the development of the virus in the plant.

Additionally, the transgenic sugar beet of the present invention expresses the manA gene (also known as pmi) from Escherichia coli. This gene encodes the phosphomannose isomerase, PMI, an enzyme which acts as a selectable marker enabling transformed plant cells to utilize mannose as a primary carbon source. Expression of pmi is driven by the heat shock protein (80) promoter (from Brassica oleracea). Untransformed sugar beet plants cannot use mannose and therefore the PMI protein acts as a selectable marker when plants are grown on media containing mannose as the sole source of carbon.

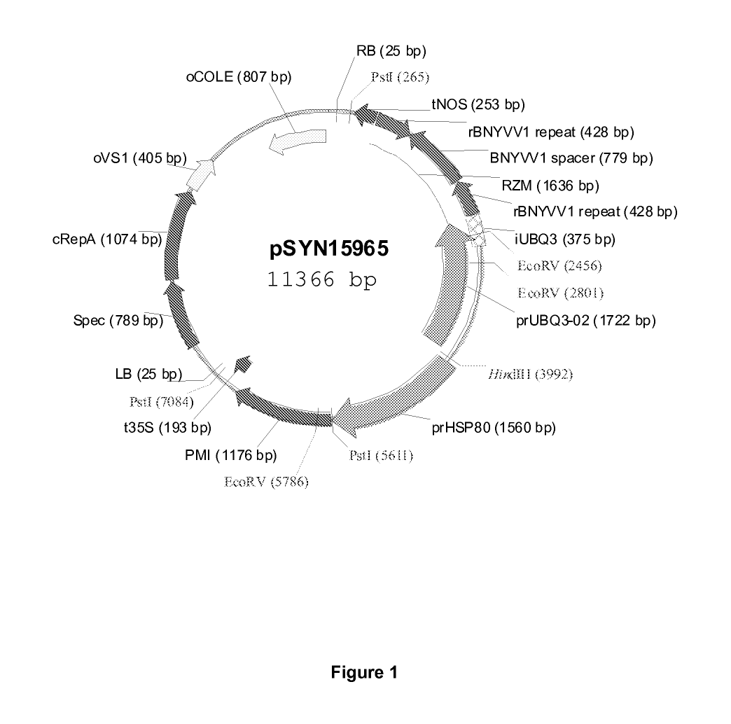

The GM RZ13 sugar beet of the present invention was generated by standard Agrobacterium tumefaciens mediated transformation techniques as described in Example 2 below. The a map of plasmid pSYN15965 (previously known as pHiNK188) used for the transformation is presented in FIG. 1. The size, function and origin of each component of pSYN15965 are described in Table 1.

In another embodiment, the present invention encompasses a sugar beet plant comprising at least a first and a second DNA sequence linked together to form a contiguous nucleotide sequence, wherein the first DNA sequence is within a junction sequence and comprises at least about 11 contiguous nucleotides selected from the group consisting of nucleotides 461-807 of SEQ ID NO: 2, nucleotides 1-237 of SEQ ID NO: 8, and the complements thereof, wherein the second DNA sequence is within the heterologous insert DNA sequence set forth in nucleotides 1-460 of SEQ ID NO: 2, nucleotides 283-484 of SEQ ID NO: 8, and the complements thereof; and wherein the first and the second DNA sequences are useful as nucleotide primers or probes for detecting the presence of sugar beet event GM RZ13 nucleic acid sequences in a biological sample. In one aspect of this embodiment, the nucleotide primers are used in a DNA amplification method to amplify a target DNA sequence from template DNA extracted from the sugar beet plant and the sugar beet plant is identifiable from other sugar beet plants by the production of an amplicon when using said first and second nucleotide primers in said DNA amplification method.

In another aspect, the present invention provides a biological sample derived from a GM RZ13 sugar beet plant, tissue, or seed, wherein the sample comprises a nucleotide sequence which is or is complementary to a nucleotide sequence that is unique to event GM RZ13, and wherein the sequence is detectable in the sample using a nucleic acid amplification or nucleic acid hybridization method. In one embodiment of this aspect, said nucleotide sequence that is unique to event GM RZ13 is or is complementary to SEQ ID NO: 1, SEQ ID NO: 2, SEQ ID NO: 7 or SEQ ID NO: 8. The sample can be derived from a seed, stalk, leave, root, flower or a part thereof. A “GM RZ13 sugar beet plant” in this context refers to a sugar beet plant of the present invention comprising a nucleotide sequence that is unique to event GM RZ13. A “GM RZ13 sugar beet tissue, or seed” means a tissue or seed of the sugar beet plant of the present invention comprising a nucleotide sequence that is unique to event GM RZ13.

In another aspect, the present invention provides an extract derived from a GM RZ13 sugar beet plant, tissue, or seed, wherein the sample comprises a nucleotide sequence which is or is complementary to a nucleotide sequence that is unique to event GM RZ13, and wherein the sequence is detectable in the sample using a nucleic acid amplification or nucleic acid hybridization method. In one embodiment of this aspect, said nucleotide sequence that is unique to event GM RZ13 is or is complementary to SEQ ID NO: 1, SEQ ID NO: 2, SEQ ID NO: 7 or SEQ ID NO: 8. The nucleic acid amplification or nucleic acid hybridization method may be any nucleic acid amplification or nucleic acid hybridization method known to the person skilled in the art. Preferably, the nucleic acid amplification or nucleic acid hybridization method is the method of detecting the presence of a nucleotide sequence that is unique to event GM RZ13 in a sample of the present invention.

In another aspect, the present invention provides a method of detecting sugar beet event GM RZ13 protein in a biological sample comprising: (a) extracting protein from a sample of sugar beet event GM RZ13 tissue; (b) assaying the extracted protein using an immunological method comprising antibody specific for the insecticidal or selectable marker protein produced by the GM RZ13 event; and (c) detecting the binding of said antibody to the insecticidal or selectable marker protein.

In another aspect, the present invention provides a method for producing a sugar beet plant resistant to at least Beet Necrotic Yellow Vein Virus comprising (a) sexually crossing a first parent sugar beet plant with a second parent sugar beet plant, wherein said first or second parent sugar beet plant comprises sugar beet event GM RZ13 DNA, thereby producing a plurality of first generation progeny plants; (b) selecting a first generation progeny plant that is resistant to at least Beet Necrotic Yellow Vein Virus; (c) selfing the first generation progeny plant, thereby producing a plurality of second generation progeny plants; and (d) selecting from the second generation progeny plants, a plant that is at least resistant to Beet Necrotic Yellow Vein Virus; wherein the second generation progeny plants comprise a nucleotide sequence that is or is complementary to a nucleotide sequence selected from the group consisting of SEQ ID NO: 1 and SEQ ID NO: 7. In a preferred embodiment of this aspect, said method is a method wherein said first or second parent sugar beet plant comprising sugar beet event GM RZ13 DNA in step a) is the Beet Necrotic Yellow Vein Virus resistant sugar beet plant of the present invention and as described hereinabove, or a plant derived from the seeds of the present invention and as described hereinabove. In this context, the expression “sugar beet plant comprising sugar beet event GM RZ13 DNA” refers to a sugar beet plant comprising a nucleotide sequence that is unique to event GM RZ13.

One skilled in the art will recognize that the sugar beet event GM RZ13 DNA of the present invention (i.e., a nucleotide sequence of the present invention that is unique to event GM RZ13) can be introgressed by breeding into other sugar beet lines comprising different transgenic or non-transgenic genotypes. For example, a sugar beet inbred comprising sugar beet event GM RZ13 DNA of the present invention (i.e., a sugar beet plant comprising a nucleotide sequence that is unique to event GM RZ13) can be crossed with a sugar beet inbred comprising the transgenic genotype of an event resistant to a different virus known to infect sugar beet plants. The resulting seed and progeny plants will have the stacked resistance traits. For example, a GM RZ13 sugar beet inbred can be crossed with a sugar beet inbred comprising the transgenic genotype of the glyphosate resistant H7-1 event (European patent application EP-A1-1597373, herein incorporated by reference). The resulting seed and progeny plants have the stacked resistance traits against both the herbicide glyphosate and the Beet Necrotic Yellow Vein Virus. Further GM traits, like herbicide resistance, insect resistance, disease resistance, transgenic plants having a phenotype of delayed or inhibited bolting, transgenic plants with changed and/or enhanced carbohydrate composition can also used for stacking with the transgenic plants of the present invention comprising sugar beet event GM RZ13 DNA (i.e., a sugar beet plant comprising a nucleotide sequence that is unique to event GM RZ13). An example of herbicide resistance is, for example, the glyphosate resistance conferred by the glyphosate resistant H7-1 event mentioned above), examples of insect resistance are resistance against feeding insects above the ground (using, for example, a VIP gene and/or a Cry gene (such as Cry1Ab (see, for example, European patent EP 0 618 976B1 (incorporated herein by reference in its entirety) and the patents belonging to the family of this patent) or VIP3 (see, for example, International patent application WO96/10083 or International patent application WO98/44137 (both incorporated herein by reference in its entirety) and the patents belonging to the family of these patent applications) and feeding pests below the ground (such as, for example, nematode resistance(against, e.g., beet cyst nematode); whereas examples of fungal resistance is the resistance either against one or against more than one fungi. Examples of plants having a phenotype of delayed or inhibited bolting are plants in which the expression of or more gene(s) selected from the group of FT, AGL20, FLC, or PRR7 is/are modified. Further Cry and VIP genes and further candidate genes for the modification of the bolting behavior of sugar beet are well known to persons skilled in the art. Sugar beet plants with modified expression of sugar beet FT genes are provided in International patent application PCT/EP2009/006319 (incorporated herein by reference in its entirety), sugar beet plants with modified expression of the AGL20 & FLC genes are provided in International patent application WO2007/122086 (incorporated herein by reference in its entirety), and sugar beet pants with modified expression of the PRR7 gene are provided in International patent application WO2009/141446 (incorporated herein by reference in its entirety). Examples of modified changed and/or enhanced carbohydrate composition are provided in International patent application WO2004/099403 and in International patent application PCT/US2009/046968 (both incorporated herein by reference in their entirety).

In preferred embodiments, the resistance to Beet Necrotic Yellow Vein Virus in the plants of the present invention is stacked with either (a) glyphosate resistance, (b) insect resistance (Vip3 or Cry1Ab or both); (c) the transgenic phenotype of delayed or inhibited bolting resulting from the modification of the expression of or more gene(s) selected from the group of FT, AGL20, FLC, or PRR7; (d) glyphosate resistance and insect resistance (Vip3 or Cry1Ab or both) as triple stack, (e) glyphosate resistance and the transgenic phenotype of delayed or inhibited bolting resulting from the modification of the expression of or more gene(s) selected from the group of FT, AGL20, FLC, or PRR7 as triple stack; (f) insect resistance (Vip3 or Cry1Ab or both) and the transgenic phenotype of delayed or inhibited bolting resulting from the modification of the expression of or more gene(s) selected from the group of FT, AGL20, FLC, or PRR7 as triple stack; or (g) herbicide resistance, insect resistance (Vip3 or Cry1Ab or both) and the transgenic phenotype of delayed or inhibited bolting resulting from the modification of the expression of or more gene(s) selected from the group of FT, AGL20, FLC, or PRR7 as quadruple stack.

In a further preferred embodiment, non-GM traits, like disease resistance or resistance against BNYVV from conventional sources (like Holly, WB41, WB42, WB151, WB169, C28, C48, C50, or Rizor, or crosses thereof) or viruses other than BNYVV can also used for stacking with the transgenic plants of the present invention comprising sugar beet event GM RZ13 DNA (i.e., a sugar beet plant comprising a nucleotide sequence that is unique to event GM RZ13)., Tolerance to pests like, for example, beet cyst nematodes, root aphids, root knot nematodes, or tolerance to fungal pests like, for example, Cercospora, Aphanomyces, Rhizoctonia, Fusarium, Ramularia, Erysipe, Peronospora, Erwinia, Sclerotium, Verticillium, Phoma, or Rust, or tolerance to viruses like, for example, Beet Curly Top Virus, Beet Yellow Virus, Beet Mild Yellow Virus, Beet Western Yellow Virus, are further traits for stacking with the transgenic plants of the present invention comprising sugar beet event GM RZ13 DNA (i.e., a sugar beet plant comprising a nucleotide sequence that is unique to event GM RZ13).

It will be further recognized that other combinations or stacks can be made with the transgenic the transgenic plants of the present invention comprising sugar beet event GM RZ13 DNA (i.e., a sugar beet plant comprising a nucleotide sequence that is unique to event GM RZ13) and thus these examples should not be viewed as limiting.

One skilled in the art will also recognize that transgenic sugar beet seed comprising a nucleotide sequence of the present invention that is unique to event GM RZ13 can be treated with various seed-treatment chemicals, including various pesticides and insecticides, to further augment the resistance against BNYVV.

The sugar beet event GM RZ13 DNA of the present invention (i.e., a nucleotide sequence of the present invention that is unique to event GM RZ13) can be introgressed in any sugar beet inbred or hybrid using art recognized breeding techniques. The goal of plant breeding is to combine in a single variety or hybrid various desirable traits. For field crops, GM and non-GM traits of interest may include the traits listed above and further agronomic traits like, for example, greater yield, and better agronomic quality. With mechanical harvesting of many crops, uniformity of plant characteristics such as germination and taproot establishment, growth rate, maturity, and root size, is important.

Plants that have been self-pollinated and selected for type for many generations become homozygous at the majority of gene loci and produce a uniform population of true breeding progeny. A cross between two different homozygous lines produces a uniform population of hybrid plants that may be heterozygous for many gene loci. A cross of two plants each heterozygous at a number of gene loci will produce a population of hybrid plants that differ genetically and will not be uniform.

Plant breeding techniques known in the art and used in a sugar beet plant breeding program include, but are not limited to, recurrent selection, backcrossing, pedigree breeding, restriction length polymorphism enhanced selection, genetic marker enhanced selection and transformation. The development of sugar beet hybrids in a sugar beet plant breeding program requires, in general, the development of homozygous inbred lines, the crossing of these lines, and the evaluation of the crosses. Pedigree breeding and recurrent selection breeding methods are used to develop inbred lines from breeding populations. Sugar beet plant breeding programs combine the genetic backgrounds from two or more inbred lines or various other germplasm sources into breeding pools from which new inbred lines are developed by selfing and selection of desired phenotypes. The new inbreds are crossed with other inbred lines and the hybrids from these crosses are evaluated to determine which of those have commercial potential. Plant breeding and hybrid development, as practiced in a sugar beet plant-breeding program, are expensive and time-consuming processes.

Pedigree breeding starts with the crossing of two genotypes, each of which may have one or more desirable characteristics that is lacking in the other or which complements the other. If the two original parents do not provide all the desired characteristics, other sources can be included in the breeding population. In the pedigree method, superior plants are selfed and selected in successive generations. In the succeeding generations the heterozygous condition gives way to (almost) homogeneous lines as a result of self-pollination and selection. Typically in the pedigree method of breeding five or more generations of selfing and selection is practiced: F1→F2; F2→F3; F3→F4; F4→F5; etc.

Recurrent selection breeding, backcrossing for example, can be used to improve an inbred line and a hybrid that is made using those inbreds. Backcrossing can be used to transfer a specific desirable trait from one inbred or source to an inbred that lacks that trait. This can be accomplished, for example, by first crossing a superior inbred (recurrent parent) to a donor inbred (non-recurrent parent), that carries the appropriate gene(s) for the trait in question. The progeny of this cross is then mated back to the superior recurrent parent followed by selection in the resultant progeny for the desired trait to be transferred from the non-recurrent parent. After five or more backcross generations with selection for the desired trait, the progeny will be homozygous for loci controlling the characteristic being transferred, but will be like the superior parent for essentially all other genes. The last backcross generation is then selfed to give pure breeding progeny for the gene(s) being transferred. A hybrid developed from inbreds containing the transferred gene(s) is essentially the same as a hybrid developed from the same inbreds without the transferred gene(s).

Elite inbred lines, that is, pure breeding, (almost) homozygous inbred lines, can also be used as starting materials for breeding or source populations from which to develop other inbred lines. These inbred lines derived from elite inbred lines can be developed using the pedigree breeding and recurrent selection breeding methods described earlier. As an example, when backcross breeding is used to create these derived lines in a sugar beet plant-breeding program, elite inbreds can be used as a parental line or starting material or source population and can serve as either the donor or recurrent parent.

As is readily apparent to one skilled in the art, the foregoing are only some of the various ways by which the inbred of the present invention can be obtained by those looking to introgress the sugar beet event GM RZ13 DNA of the present invention (i.e., a nucleotide sequence of the present invention that is unique to event GM RZ13) into other sugar beet lines. Other means are available and known to the person skilled in the art, and the above examples are illustrative only.

A single cross corn hybrid results from the cross of two inbred lines, each of which has a genotype that complements the genotype of the other. The hybrid progeny of the first generation is designated F1. In the development of commercial hybrids in a sugar beet plant-breeding program, only the F1 hybrid plants are sought. Preferred F1 hybrids are more vigorous than their inbred parents. This hybrid vigor, or heterosis, can be manifested in many polygenic traits, including increased vegetative growth and increased yield.

The development of a sugar beet hybrid in a sugar beet plant breeding program involves three steps: (1) the selection of plants from various germplasm pools for initial breeding crosses; (2) the selfing of the selected plants from the breeding crosses for several generations to produce a series of inbred lines, which, although different from each other, breed true and are highly uniform; and (3) crossing the selected inbred lines with different inbred lines to produce the hybrid progeny (F1). During the inbreeding process in sugar beet, the vigor of the lines decreases. Vigor is restored when two different inbred lines are crossed to produce the hybrid progeny (F1). An important consequence of the homozygosity and homogeneity of the inbred lines is that the hybrid between a defined pair of inbreds will always be the same. Once the inbreds that give a superior hybrid have been identified, the hybrid seed can be reproduced indefinitely as long as the homogeneity of the inbred parents is maintained.

A single cross hybrid is produced when two inbred lines are crossed to produce the F1 progeny. A double cross hybrid is produced from four inbred lines crossed in pairs (A×B and C×D) and then the two F1 hybrids are crossed again (A×B)×(C×D). A three-way cross hybrid is produced from three inbred lines where two of the inbred lines are crossed (A×B) and then the resulting F1 hybrid is crossed with the third inbred (A×B)×C. Much of the hybrid vigor exhibited by F1 hybrids is lost in the next generation (F2). Consequently, seed from hybrids is not used for planting stock.

In hybrid seed production it is preferred to eliminate or inactivate pollen production by the female parent. Incomplete removal or inactivation of the pollen provides the potential for self-pollination. This inadvertently self-pollinated seed may be unintentionally harvested and packaged with hybrid seed. Once the seed is planted, it is possible to identify and select these self-pollinated plants. These self-pollinated plants will be genetically equivalent to the female inbred line used to produce the hybrid. Typically these self-pollinated plants can be identified and selected due to their decreased vigor. Female selfs are identified by their less vigorous appearance for vegetative and/or reproductive characteristics. Identification of these self-pollinated lines can also be accomplished through molecular marker analyses.

However, simple and efficient pollination control systems exist which ensure utilizing heterosis by excluding self-pollination in commercial hybrid seed production. If one of the parents is a self-incompatible (SI), cytoplasmic male sterile (CMS) or nuclear male sterile (NMS) plant that is not able to self-pollinate or is incapable of producing pollen, only cross pollination will occur. By eliminating the pollen of one parental variety in a cross, a plant breeder is assured of obtaining hybrid seed of uniform quality, provided that the parents are of uniform quality and the breeder conducts a single cross. Cytoplasmic male sterility (CMS) is a maternally inherited phenomenon, the genetic determinants of which are located in the genome of the cytoplasmic organelles, the mitochondria. Such plants are severely impaired in their ability to produce functional pollen grains. Restorer genes for CMS systems are dominant nuclear genes, which suppress male sterile effects of the cytoplasm. The expression of male sterility in CMS plants is the result of incompatibility between recessive nuclear gene and male sterile specific cytoplasmic genome.

In another aspect, the present invention provides a method for producing Beet Necrotic Yellow Vein Virus resistant sugar beet hybrid seed. Such a method comprises: (a) providing a Beet Necrotic Yellow Vein Virus resistant sugar beet line as a first parent line, (b) providing a second sugar beet line having a different genotype as a second parent line; (c) allowing the plants of the first parent line of step (a) and the plants of the second parent line of step (b) to pollinate each other, let the seed develop, and harvest the hybrid seed, wherein the harvested hybrid seeds are seeds of a Beet Necrotic Yellow Vein Virus resistant sugar beet plant.

In a preferred embodiment, a CMS system is applied for production of the hybrid sugar beet plants of the present invention. In such a system a male sterile CMS line is used as female parent that is pollinated by a male fertile line used as male parent. The nucleic acid molecule of the present invention that is unique to sugar beet event GM RZ13 can be present invention can be present in both the CMS male sterile (female) parent line or the male fertile (male) parent line or even both. Preferably, the nucleic acid molecule of the present invention that is unique to sugar beet event GM RZ13 is kept on the male sterile side in order to avoid GM contaminations via the pollen containing the trait shed by the male parent. Thus, in a preferred embodiment the male sterile CMS sugar beet parental line provided in step a) or b) of the above method is an inbred sugar beet line comprising a nucleotide sequence of the present invention that is unique to event GM RZ13. Further, in such a system both parents can be transgenic plants.

Thus, another preferred embodiment of said method is a method for producing Beet Necrotic Yellow Vein Virus resistant sugar beet hybrid seed comprising the steps of: (a) providing a Beet Necrotic Yellow Vein Virus resistant sugar beet line as a first parent line, (b) providing a second sugar beet line having a different genotype as a second parent line, wherein one of the parent lines used in step a) or step b) is a male sterile CMS line and wherein the other parent line is male fertile; and (c) allowing the plants of the male fertile parent line to pollinate the flowers of the male sterile parent line, let the seed develop, and harvest the hybrid seed, wherein the harvested hybrid seeds are seeds of a Beet Necrotic Yellow Vein Virus resistant sugar beet line, preferably hybrid seed of a Beet Necrotic Yellow Vein Virus resistant sugar beet line comprising the nucleic acid molecule of the present invention that is unique to sugar beet event GM RZ13.

In yet another preferred embodiment of this aspect, the Necrotic Yellow Vein Virus resistant sugar beet line used as a first parent line in step (a) is a Beet Necrotic Yellow Vein Virus resistant inbred sugar beet line comprising the nucleic acid molecule of the present invention that is unique to sugar beet event GM RZ13. In a further embodiment of this aspect, the second parental line is selected from the group consisting of (a) an inbred sugar beet plant line resistant to at least Beet Necrotic Yellow Vein Virus having a different genotype but comprising a nucleic acid molecule of the present invention that is unique to sugar beet event GM RZ13; (b) an inbred sugar beet plant line resistant to at least Beet Necrotic Yellow Vein Virus which originates from a naturally occurring source selected from the group comprising the Holly source, WB41, WB42, WB151, WB169, C28, C48, C50, or Rizor, or crosses thereof; and (c) an inbred sugar beet plant line having no resistance to the Beet Necrotic Yellow Vein Virus.

The expression “inbred sugar beet plant line resistant to at least Beet necrotic yellow vein virus having a different genotype but comprising a nucleotide sequence of the present invention that is unique to sugar beet event GM RZ13” means a sugar beet plant that comprises a nucleotide sequence of the present invention but otherwise differs by at least one gene or trait. Such inbred sugar beet plant may comprise further GM or non-GM traits as listed above. The inbred sugar beet plant in step a) can be the Beet Necrotic Yellow Vein Virus resistant sugar beet plant of the present invention and as described hereinabove or a plant derived from the seeds of a Beet Necrotic Yellow Vein Virus resistant sugar beet plant of the present invention and as described hereinabove.

The term “originates” as used in the context of the naturally occurring sources for resistance against the Beet Necrotic Yellow Vein Virus selected from the group comprising the Holly source, WB41, WB42, WB151, WB169, C28, C48, C50, or Rizor, or crosses thereof, refers to BNYVV resistant sugar beet plants the resistance of which against BNYVV is derived from conventional sugar beet lines or sugar beet wild types which are of non-transgenic origin. These conventional resistances are known to the person skilled in the art. The Holly resistance goes back the genes present in the Holly source (Lewellen et al., 1987) in which the major dominant gene Rz1 confers resistance to BNYVV (Pelsy and Merdinoglu, 1996; Scholten et al., 1996). The further conventional BNYVV resistant sources WB41 and WB42 originate from two plants of Beta vulgaris ssp maritima collected in Denmark (Lewellen et al., 1987; Whitney, 1989), whereas the conventional rhizomania resistant sugar beet line C48 was developed from crosses of WB41 and WB42 to line C37. WB151, WB169, C28, and C50 as sources of Rhizomania resistance are described by Lewellen (1995).

In general, the second parent line used for the hybrid production can also be a BNYVV resistant sugar beet plant line like, for example, a sugar beet plant of the present invention comprising the nucleic acid molecule of the present invention that is unique to sugar beet event GM RZ13. Preferably, the first parent line and the second parent line employed in the production of the hybrid seed are based on genetically diverse backgrounds. Genetic distance can be measured by the use of molecular markers as described for example in Knaak (1996). However, the second parent line could also be a sugar beet inbred comprising the transgenic genotype of the glyphosate resistant H7-1 event (European patent application EP-A1-1597373, herein incorporated by reference). The resulting hybrid seed will contain the stacked resistance traits against both the herbicide glyphosate and the Beet Necrotic Yellow Vein Virus. The goal of plant breeding is to combine in a single variety or hybrid various desirable traits. Further traits, like GM and non-GM traits of interest including, but not limited to the traits listed above and further agronomic traits like, for example, greater yield, and better agronomic quality can also be comprised in the second parent line for stacking with the nucleic acid molecule of the present invention that is unique to sugar beet event GM RZ13 in the hybrid seed. It will be further recognized that other combinations or stacks can be made with the nucleic acid molecule of the present invention that is unique to sugar beet event GM RZ13 and thus these examples should not be viewed as limiting.

Another preferred embodiment of the present invention relates to hybrid seed of a BNYVV resistant sugar beet plant. In one aspect of the present invention said hybrid seed is produced by the method for producing BNYVV resistant sugar beet hybrid seed of the present invention. In yet another aspect of the present invention a BNYVV resistant sugar beet hybrid plant is provided that is produced by growing the hybrid seed of the present invention. Preferably, this hybrid plant comprises a nucleic acid molecule of the present invention that is unique to sugar beet event GM RZ13. A further preferred embodiment of the present invention relates to a part of said BNYVV resistant sugar beet plant hybrid plant of the present invention. Preferably said part is selected from the group comprising seeds, embryos, microspores, zygotes, protoplasts, cells, ovules, pollen, taproots, cotyledons, or other reproductive or vegetative parts.

In another aspect, the present invention provides the use of a BNYVV resistant sugar beet plant of the present invention or cells or tissues thereof, a biological sample or an extract thereof in a method selected from the group comprising of methods of sugar production, methods of aerobic fermentation and methods of anaerobic fermentation. Preferably, said use is the use of a BNYVV resistant sugar beet plant of the present invention or cells or tissues thereof, a biological sample or an extract thereof in a method of producing sugar. The method for producing sugar can be any method known to person skilled in the art. In one embodiment of this aspect, the BNYVV resistant sugar beet plant used in a method for producing sugar as well as the cells or tissues, the biological sample or the extract are obtained from plants produced from the sugar beet seed comprising the nucleic acid molecules of the invention deposited at NCIMB under the accession No. 41601. The term “plants produced from the sugar beet seed” in this context refers to sugar beet plants grown from the seeds as well as to hybrids produced by using sugar beet plants grown from the seeds. In a further preferred embodiment, the present invention also relates to a method of using a BNYVV resistant sugar beet plant of the present invention or cells or tissues thereof, a biological sample or an extract thereof in a method selected from the group comprising of methods of sugar production, methods of aerobic fermentation and methods of anaerobic fermentation.

Further encompassed are methods for producing sugar, wherein a BNYVV resistant sugar beet plant, or cells or tissues thereof, a biological sample or an extract of the present invention is processed to produce sugar. Further, sugar is provided by the present invention that is produced by the method of producing sugar of the present invention. The method for producing sugar can be any conventional method for producing sugar known to person skilled in the art. In one embodiment of this aspect, said BNYVV resistant sugar beet plant, the cells or tissues are obtained from plants produced from the sugar beet seed comprising the nucleic acid molecules of the invention deposited at NCIMB under the accession No. 41601. Further, the biological sample or the extract is a biological sample or extract obtained from this plant material. The term “plants produced from the sugar beet seed” in this context refers to sugar beet plants directly grown from the seeds as well as to hybrids produced by using sugar beet plants grown from the seeds.

In another aspect the present invention encompasses a method for producing one or more biofuel(s) selected from the group comprising ethanol, butanol, biogas and/or biodiesel, by processing a BNYVV resistant sugar beet plant, or cells or tissues thereof, or a biological sample or an extract of the present invention to produce the one or more biofuel(s). The biofuel can be any biofuel produced by aerobic or anaerobic fermentation of plant material. A non-limiting example of a biofuel obtained by aerobic fermentation is bioethanol or butanol. Biofuels that can be obtained by anaerobic fermentation include, but are not limited to biogas and/or biodiesel. Methods of aerobic and/or anaerobic fermentation are known to the person skilled in the art. Further encompassed by the present invention are biofuels selected from the group comprising ethanol, butanol, biogas and/or biodiesel as produced by the method for producing one or more biofuel(s) or the present invention.

“Fermentation” can refer to the process of transforming an organic molecule into another molecule using a micro-organism. For example, “fermentation” can refer to aerobic transforming sugars or other molecules from plant material, such as the plant material of the present invention, to produce alcohols (e.g., ethanol, methanol, butanol); organic acids (e.g., citric acid, acetic acid, itaconic acid, lactic acid, gluconic acid); ketones (e.g., acetone), amino acids (e.g., glutamic acid); gases (e.g., H₂ and CO₂), antibiotics (e.g., penicillin and tetracycline); enzymes; vitamins (e.g., riboflavin, B12, beta-carotene); and/or hormones. Fermentation can include fermentations used in the consumable alcohol industry (e.g., beer and wine), dairy industry (e.g., fermented dairy products), leather industry, and tobacco industry. Thus, fermentation includes alcohol fermentation. Fermentation also includes anaerobic fermentations, for example, for the production of biogas. Fermenting can be accomplished by any organism suitable for use in a desired fermentation step, including, but not limited to, bacteria, fungi, archaea, and protists. Suitable fermenting organisms include those that can convert mono-, di-, and trisaccharides, especially glucose and maltose, or any other biomass-derived molecule, directly or indirectly to the desired fermentation product (e.g., ethanol, butanol, etc.). Suitable fermenting organisms also include those which can convert non-sugar molecules to desired fermentation products. Such organisms and fermentation methods are known to the person skilled in the art.

As described hereinabove, breeding for resistance against BNYVV is limited by the availability and durability of resistant sources in the germplasm pool of sugar beet. In the present invention, RNA silencing was successfully exploited to engineer resistance against BNYVV by the transgenic expression of a 428 by inverted repeat derived from the BNYVV replicase gene (RNA1 of the viral genome). The transgenic resistance was stably inherited over generations and shown to be efficient not only in greenhouse trials, but also in the field, as shown herein in Examples 5 to 8.

In fact, the resistance in the transgenic plants of the present invention alone or in addition to the resistance obtained from Holly has been shown to be superior to the resistance conferred by conventional resistances from the sources Holly and C48 (see Examples 5 to 7 below) even when challenged with BNYVV of different types and origin including highly aggressive strains. Whereas the partial resistance observed in BNYVV resistant sugar beet lines containing conventional resistance from Holly is partially lost in hybrids obtained from using this line (i.e., the resulting hybrids become significantly more susceptible to BNYVV), the transgenic event GM RZ13 of the present invention shows a strong resistance in all tested soils even in hybrid plants obtained from using plant material containing event GM RZ13 (see Example 8 below). The observed resistance is significantly stronger than the resistance in any one of conventional hybrids. Thus, the transgenic plants of the present invention containing event GM RZ13 show consistently improved reduction in virus concentrations compared to native trait approaches (i.e., resistances obtained from the Holly source of from C48) in tests using diverse sources of infected soils containing the different known types of BNYVV (see Example 8 below). Further, the new highly pathogenic strains are also controlled by the GM RZ13 event. No negative effect on the sugar content, the root weight and the juice purity (with regards to characteristics like the content of K, Na, and Amino-N) has been observed in plants containing event GM RZ13 in trials over several years in different locations in the USA and Europe.

Thus, the transgenic hybrids of the present invention are of high value in all areas in the world with rhizomania infested soils and especially in areas with high infection pressure or with deviant types of the BNYVV virus, where the conventional Holly resistance or other conventional resistance sources currently available are not strong enough or have already been broken by the virus.

The following examples are intended solely to illustrate one or more preferred embodiments of the invention and are not to be construed as limiting the scope of the invention.

### EXAMPLES

### Example 1

### Vector Construction

Sugar beet roots infected with the B-type of BNYVV were collected from the Harting region in Germany and total RNA was extracted using the RNeasy Plant Mini kit from Qiagen following the supplier's instruction. BNYVV RNA1 encoding the BNYVV replicase was converted into cDNA using the SUPERSCRIPT™ II RNase H-reverse transcriptase from Life Technologies, essentially as described by the supplier, and using oligonucleotide HiNK285 (5′-TCGTAGAAGAGAATTCACCCAAACTATCC-3′, SEQ ID NO: 28) as reverse primer. Subsequently, the ultimate 1.4 kb of RNA1 spanning the region harboring the GDD motif to the 3′ UTR was amplified using primer HiNK283 (5′-AAGAATTGCAGGATCCACA-GGCTCGGTAC-3′, SEQ ID NO: 29) in combination with HiNK285 in a standard PCR reaction. The sequence of BNYVV RNA1 with accession number X05147 (Bouzoubaa et al., 1987) was used as reference for the design of the various oligonucleotides, recognition sequences of restriction enzymes BamHI and EcoRI in the primer sequences given above are underlined. The obtained PCR fragment was fused to a second amplification fragment of 0.4 kb spanning the GDD region only that was amplified using primers HiNK283 and HiNK284 (5′-TTCCAACGAATTCGGTCTCAGACA-3′, SEQ ID NO: 30). Both fragments were ligated at the EcoRI sites present at primers HiNK284 and HiNK285 such that both GDD motifs were in opposite direction resulting in the formation of an inverted repeat (the construct including the inverted repeat and the sequence from RNA1 of BNYVV is referred to as RZM in Table 1 and FIG. 1). The inverted repeat thus consists of the 0.4 kb GDD region interrupted by the 3′ end of RNA1. The Ubi3 promoter from Arabidopsis thaliana including its cognate 5′ UTR and intron 1 (Norris et al., 1993) was cloned upstream of the inverted repeat to drive constitutive expression. Polyadenylation occurred at the nos terminator. The entire cassette was introduced onto the T-DNA of a proprietary binary vector, next to the selectable marker gene consisting of the phosphomannose isomerase (PMI) gene for mannose selection (Reed et al., 2001), yielding binary vector pSYN15965 (previously known as pHiNK188) (see FIG. 1). The PMI gene is driven by the heat shock protein (80) promoter from Brassica oleracea and is followed by a 35S terminator. The constituents of pSYN15965 are further listed in Table 1.

TABLE 1
Size, function, and source of the
constituents in vector pSYN15965.

| | Size of | Intended | |
|---|---|---|---|
| DNA sequence | sequence | function | Source and reference |
| Ubiquitin 3 | 1.7 kb | Promoter incl. | Arabidopsis thaliana |
| | | first intron | NORRIS et al., 1993 |
| RZM | 1.6 kb | Resistance | BNYVV |
| | | to BNYVV | BOUZOUBAA et al., 1987 |
| Nos | 0.3 kb | Terminator | Agrobacterium tumefaciens |
| | | | FRALEY et al. 1983 |
| Hsp80 | 1.5 kb | Promoter | Brassica sp. |
| | | | BRUNKE & WILSON, |
| | | | 1993 |
| PMI | 1.2 kb | Selectable | Escherichia coli |
| | | marker | JOERSBO et al., 1998 |
| 35S | 0.2 kb | Terminator | Cauliflower Mosaic Virus |
| | | | ODELL et al., 1985 |

### Example 2

### Transformation and In Vitro Selection of Transgenic Shoots

A conventional, rhizomania susceptible breeding line from Syngenta Seeds AB, Landskrona, Sweden, referred to as G018 was used as acceptor for transformation. Sugar beet seeds were surface-sterilized and germinated in vitro. Agrobacterium-mediated transformation of cotyledonary node explants using mannose isomerase as selectable marker gene was carried out essentially as described by Joersbo et al. (1998). Selection of transgenic sugar beet shoots was started 2-4 days after co-cultivation by gradually substituting sucrose by D-mannose to a final concentration of 12 g/L as predominant carbohydrate source in the regeneration medium. The selective regeneration yielded transgenic shoots in 12-15 weeks. To verify that the shoots were transgenic, several leaf tips from each of the regenerated shoots were harvested and the phosphomannose isomerase (PMI) activity measured using a coupled enzyme assay described by Joersbo et al. (1998). Clonal propagation and rooting of transgenic shoots were carried out on standard MS-medium (Murashige & Skoog, 1962) supplemented with 0.25 mg/L BA (6-benzylamino purine) for propagation and with 5 mg/L IBA (indole-3-butyric acid) for root induction. Propagation and rooting were performed while maintaining mannose selection at a concentration of 12 g/L to eliminate chimeric plants. Each primary regenerant (R₀ plant) was propagated in vitro to deliver three to six R₀ plants that subsequently were rooted in a sandy soil in a growth chamber. After rooting the plants were moved to the greenhouse for phenotypic testing. The R₀ plants were crossed to conventional or rhizomania resistant genotypes homozygous for Holly, using the R₀ plants as females.

### Example 3

### Analysis of Transgene mRNA and siRNA Accumulation

Seedlings obtained in Example 2 were sown in sterile sand and subsequently transplanted into tubes containing 0.25 L sterile sand or soil infested with the B-type of BNYVV. For the detection of transgene mRNA and siRNA, root samples (0.2 g per plant) were collected at 0, 7, 14, 21 and 28 days post-transplantation (post-inoculation, dpi). In some cases roots from a few plants were pooled to achieve a sample weight of 0.2 g. Control samples containing transgene-specific siRNA and mRNA were generated by infiltrating Agrobacterium tumefaciens strain EHA101 containing pHiNK188 into Nicotiana benthamiana leaves according to the method of Johansen and Carrington (2001).

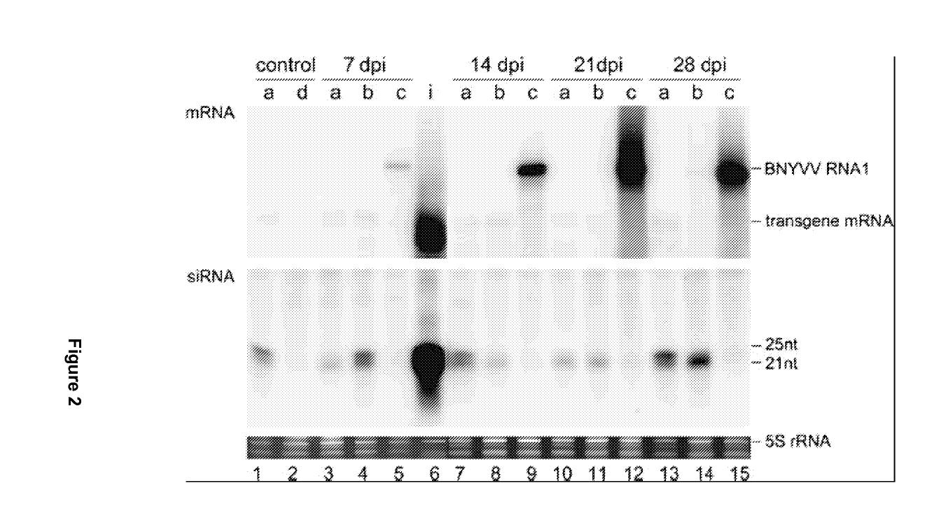

Total RNA was extracted from sugar beet roots or the infiltrated N. benthamiana leaves using a previously described protocol (Kreuze et al., 2005). The fraction of high molecular weight (HMW) RNA was used for the detection of transgene mRNA and viral RNA. The low molecular weight (LMW) RNA fraction was used for siRNA detection (Kreuze et al., 2005). Sense and antisense RNA probes labeled with [α-^{32P}] UTP were generated using the RiboMAX kit from Promega according to the supplier's protocol. For northern blot analysis, 10 pg HMW RNA was loaded onto formaldehyde (1.2% agarose) gels and separated by electrophoresis. For detection of siRNA, 15 pg LMW RNA was mixed in a 1:1 ratio with Tris-borate-EDTA-urea sample buffer (Bio-Rad), incubated at 95° C. for 5 min and separated in a 15% polyacrylamide gel (TBE-7 M Urea Ready Gel, Bio-Rad) until the bromophenol blue dye had migrated to the bottom of the gel. The separated RNAs were transferred to Hybond-N nylon membrane (Amersham) by capillary blotting. Blots were UV-cross-linked (1,200 μJ cm⁻², UV cross linker, Amersham), prehybridized, hybridized at 55° C. and 37° C. for HMW and LMW RNA, respectively, and washed as described in Kreuze et al. (2005). The washed membranes were wrapped in polyethylene film, and exposed into an exposure cassette for 1-48 h. The cassette was subsequently scanned with a Molecular Imager FX from Molecular Dynamics (see FIG. 2).

The Northern blot analysis of the HMW RNA extracted from resistant roots as described above revealed extremely low accumulation levels of transgene mRNA (FIG. 2, upper panel), which suggested that the transgene was post-transcriptionally silenced. This was confirmed by detecting transgene-homologous siRNA in significant amounts in the LMW RNA fraction from roots of all plants analyzed, except for the non-transgenic plants (FIG. 2, lower panel). Silencing of the transgene and siRNA accumulation strongly correlated with resistance, as BNYVV RNA was detected only in the roots of non-transgenic control plants and never in the resistant roots (FIG. 2, upper panel: lanes 5, 9, 12, and 15).

### Example 4

### Phenotypic Testing of R₀ Plants for Rhizomania Resistance

Seeds were germinated in sterile sand and the developing plants were delivered to the greenhouse after rooting. Upon acclimatization to the greenhouse conditions, plants were challenged by potting a subset of the transgenic R₀ clones into soil infested with a B-type BNYVV isolate from Germany. The infested soil was diluted 1:1 with sand. Plants were grown in tubes containing 0.25 L of soil; in experiments with growing periods longer than 2 months, the volume of the pots was 2.0 L. All experiments were performed in the greenhouse with day and night temperatures of 22° C. and 20° C., respectively, and a 16 h photoperiod. Plants in populations segregating for the transgenic locus were tested for PMI activity or by means of PCR in order to segregate transgenic and non-transgenic progeny plants. Non-transgenic segregants served as susceptible controls. All these susceptible control plants underwent the same in vitro regeneration protocol, except that they were not selected for mannose assimilation. After 4 weeks the challenged plants were lifted and sap was extracted from the roots and the virus titers determined by means of ELISA (Clark et al., 1977; Gidner et al., 2005). A conventional hybrid known to be highly susceptible to BNYVV was included in all experiments as susceptible control. Conventional rhizomania resistant hybrids from Syngenta Seeds carrying the resistance sources from Alba, Rizor, C48 and/or Holly, were included in the experiments as reference. All experiments were randomized according to a randomized block design with 2-4 replicates.

Out of 47 independent R₀ clones tested, 27 showed significant levels of resistance with virus titers below or equal to those measured in resistant control plants. All plants of the susceptible control had high concentrations of BNYVV. Resistant R₀ clones were selected and taken to the next generation by cross-pollination of the remaining R₀ plants that were maintained in sterilized soil. Selected plants were cross-pollinated with a susceptible genotype as well as a homozygous Holly resistant genotype to deliver progeny segregating for the transgenic resistance in a susceptible or a heterozygous Holly background.

### Example 5

### Phenotypic Characterization of the Transgenic Rhizomania Resistance

To determine the spectrum and degree of resistance in comparison to natural sources of rhizomania resistance, transgenic T₁ plants were tested in soils containing different types of BNYVV.

Tests were run in a climate chamber, wherein the day temperatures were kept at +17 to +19° C. and the night temperature was set to +17° C. Due to the heat of the lamps in the climate chamber the day temperature raised to about +21to +22° C. The plants were not watered in excess; the day length was set to 16 h. The tested plants were progenies of resistant R₀ plants that all carried a single copy of the T-DNA as determined by Southern blot analysis (data not shown). Genotyping of the T1 plants by means of the PMI assay or PCR revealed a 1:1 segregation ratio as expected for single copy insertions and allowed for the identification of transgenic and non-transgenic segregants. The non-transgenic segregants served as susceptible controls.

In a first approach soils originating from Spain (containing A-type BNYVV), Germany (containing B-type BNYVV), and from the Pithiviers region in France (containing P-type BNYVV), respectively, were used. Duncan's multiple range test showed that the virus titers in the transgenic T₁ progenies were significantly lower compared to the titers in resistant hybrids carrying conventional resistance when tested in the B-type soil (Table 2A), except for progeny 2 (statistical group CD) that was not significantly different from the combination of C48×Holly (statistical group C), but superior to all the other conventional resistance sources. When compared to each other in a susceptible or in the Holly background, the transgenic progenies were not significantly different (statistical groups DE and E), except for progeny 2 in the susceptible background again (statistical group CD) that appeared slightly less resistant. Contrary to progeny 2, the resistance levels in progenies 1 and 3 showed no significant improvement when stacked with conventional Holly resistance, probably because of the extreme resistance levels conferred by these two transgenic events alone.

The results of the statistical analysis applying ANOVA and Duncan's multiple range test are shown in Tables 2A to 2C below. Virus titers measured in entries that share the same letter are not significantly different with a confidence of at least 95%. The susceptible control consisted of non-transgenic segregants that did not inherit the transgenic locus. For all tables, virus content is expressed in log₁₀ ng ml⁻¹ in root sap of sugar beet plants challenged with BNYVV infection.

TABLE 2A
T₁ progeny plants grown in green-house for
1 month in soil infested with B-type BNYVV

| | Mean log ng | Number of | Duncan |
|---|---|---|---|
| Plants of | BNYVV ml⁻¹ | plants | grouping |
| Holly × susceptible | 2.70 | 16 | A |
| Holly × Holly | 2.15 | 20 | B |
| Holly × Rizor | 2.01 | 20 | B |
| C48 × Holly | 0.92 | 20 | C |
| T1 progeny 2 | 0.65 | 12 | C D |
| T1 progeny 1 + Holly | 0.45 | 23 | D E |
| T1 progeny 3 | 0.43 | 20 | D E |
| T1 progeny 1 | 0.32 | 20 | D E |
| T1 progeny 2 + Holly | 0.21 | 18 | E |
| T1 progeny 3 + Holly | 0.19 | 22 | E |

Not included in ANOVA

| | | |
|---|---|---|
| Susceptible control | >2.95 | 20 |

In order to test the transgenic resistance against the more virulent P-type, the T₁ progenies of 11 R₀ clones were challenged in soil collected from the Pithiviers area in France. Despite the higher titers measured in the P-type compared to the B-type soil, the transgenic progenies, whether or not combined with Holly, showed significantly lower BNYVV contents compared to the combination of C48×Holly, the strongest combination of conventional resistance sources (Table 2B). This result proves that the transgenic resistance is efficacious even under pressure of highly virulent BNYVV strains, especially when noticing that all homozygous Holly resistant control plants showed virus titers similar to the susceptible controls.

TABLE 2B
T₁ progeny plants grown in green-house for
1 month in soil infested with P-type BNYVV

| | Mean log ng | Number of | Duncan |
|---|---|---|---|
| Plants of | BNYVV ml⁻¹ | plants | grouping |
| C48 × Holly | 1.90 | 19 | A |
| T1 progeny 1 | 1.44 | 10 | B |
| T1 progeny 4 | 1.24 | 6 | B |
| T1 progeny 5 | 1.21 | 9 | B |
| T1 progeny 6 | 1.11 | 8 | B |
| T1 progeny 7 | 1.10 | 5 | B |
| T1 progeny 8 | 1.09 | 13 | B |
| T1 progeny 9 | 1.08 | 8 | B |
| T1 progeny 10 | 0.95 | 9 | B |
| T1 progeny 11 | 0.86 | 5 | B |
| T1 progeny 12 | 0.63 | 16 | B |
| T1 progeny 13 | 0.59 | 14 | B |
| T1 progeny 1 + Holly | 1.18 | 12 | B |

Not included in ANOVA

| | | |
|---|---|---|
| Susceptible control | >2.95 | 20 |
| Holly × Holly | >2.95 | 20 |

In a further study, 10 transgenic and 10 non-transgenic plants were grown in soil from Spain that contained BNYVV of the A-type. As a control, the same set and number of plants were grown in B-type soil. The sap samples of transgenic plants grown in A and B-type soil contained 1.12 and 1.65 log₁₀ ng BNYVV ml⁻¹, respectively (data not shown). The higher virus titer observed in resistant plants in this experiment is anticipated to be caused by exceptionally high greenhouse temperatures and consequently higher watering regimes, which most probably rendered the fungal vector more active compared to other experiments made in B-type soil leading to higher challenging rates. All non-transgenic plants, however, showed much higher virus titers of more than 2.95 log₁₀ ng BNYVV ml⁻¹. These results indicate that the transgenic resistance is efficient against the A-type, as it is to the B-type.

All plants in the experiments described above were pulled up and analyzed after 1 month of growth in rhizomania-infested soil. To evaluate if the transgenic resistance was durable over a period of time corresponding to the growing season of a sugar beet crop in the field, T₂ progenies from two independent R₀ clones were grown for 5 months in soils infested with the B-type or P-type of BNYVV. Both transgenic events showed significantly lower virus content compared to the homozygous Holly control, but were not significantly different from homozygous C48 when grown in the B-type soil (Table 2C). In the soil containing the P-type, the control plants homozygous for Holly became all highly infected reaching virus titers of greater than 2.95 log₁₀ ng ml⁻¹. The transgenic plants, however, maintained their resistance levels and showed significantly lower virus titers compared to both homozygous Holly and homozygous C48 plants (Table 2C). Taken together, these results show that the transgenic resistance is durable over typical cropping periods of as long as 5 months in soils containing the B-type or the more virulent P-type when Holly no longer provides adequate protection.

TABLE 2C
T₂ progeny plants grown in green-house for 5 months
in soil infested with either B-type or P-type BNYVV

| | | | |
|---|---|---|---|
| | Mean log ng | Number of | Duncan |
| Plants of | BNYVV ml⁻¹ | plants | grouping |

B-type BNYVV

| | | | |
|---|---|---|---|
| Holly × Holly | 1.88 | 10 | A |
| C48 × C48 | 0.91 | 10 | B |
| T2 progeny 1 | 0.59 | 10 | B |
| T2 progeny 2 | 0.49 | 10 | B |

Not included in ANOVA

| | | |
|---|---|---|
| Susceptible control | >2.95 | 10 |

P-type BNYVV

| | | | |
|---|---|---|---|
| C48 × C48 | 1.41 | 10 | A |
| T2 progeny 1 | 0.65 | 10 | B |
| T2 progeny 2 | 0.61 | 10 | B |

Not included in ANOVA

| | | |
|---|---|---|
| Susceptible control | >2.95 | 10 |
| Holly × Holly | >2.95 | 10 |

In a second approach also a soil from the Imperial Valley (USA) is used. Basis for the second approach have been several reports about BNYVV strains breaking the resistance in “Holly” materials. It is not yet understood if certain sequences in the genome cause the higher aggressiveness in the isolates. In this green-house study, soils were used where the resistance in “Holly” seems to be broken. These soils with the aggressive BNYVV isolates come from Spain (A-type), Imperial Valley, USA (A-type) and Pithiviers, France (P-type), respectively. For comparison a soil from Germany containing a “normal” strain of BNYVV (B-Type; showing no exceptional aggressiveness) was used.

Plants used in this approach were the transgenic event GM RZ 13 alone or crossed with a conventional line carrying the “Holly” resistance. Conventional Holly hybrids and hybrids with a combined resistance of “Holly and C48” were used as controls. Conventional hybrids having no resistance served as susceptible control. The plants were grown and analyzed as described above.

Results with the soil from Spain highly infested with an aggressive strain of A-type BNYVV show that plant material containing the GM RZ13 event also containing the resistance from conventional Holly has very low BNYVV content that is significantly lower than those in the plants containing the conventional resistance only (Table 3A).

TABLE 3A
Plants grown in soil from Spain infested with A-type BNYVV

| | Mean log ng | Number of | Duncan |
|---|---|---|---|
| Plants of | BNYVV ml⁻¹ | plants | grouping |
| Conventional Holly | 3.91 | 9 | A |
| Conventional Holly | 3.9 | 8 | A |
| Holly + C48 | 3.39 | 9 | A |
| GM RZ13 + Holly | 1.83 | 8 | B |

Results obtained using soil from the Imperial Valley (USA) or soil from the Pithiviers area in France, respectively, similarly show that plant material containing the GM RZ13 alone or in addition to the resistance obtained from Holly has very low BNYVV content even in these soils (Tables 3B and 3C). The virus titer is significantly lower than the BNYVV titers in the plant material containing the conventional Holly resistance. One conventional Holly line (termed Holly+C48 2 in Tables 3B and 3C below) also showed resistance, but in a hybrid obtained from this line which also contained the resistance from C48 (termed Holly+C48 1 in Table 3B and 3B below) the resistance was significantly reduced. The rate of infestation with an aggressive strain of A-type BNYVV in the soil from the Imperial Valley is extremely high; however, the soil does not contain Beet Soil Borne Mosaic Virus (BSBMV).

TABLE 3B
Plants grown in soil from Imperial Valley
(USA) infested with A-type BNYVV

| | Mean log ng | Number of | Duncan |
|---|---|---|---|
| Plants of | BNYVV ml⁻¹ | plants | grouping |
| Conventional Holly 1 | 4.54 | 19 | A |
| Conventional Holly 2 | 4.39 | 19 | A |
| Conventional Holly 3 | 4.34 | 9 | A B |
| Susceptible Control | 4.3 | 10 | A B |
| Holly + C48 1 | 4.00 | 20 | B |
| Holly + C48 2 | 2.21 | 20 | C |
| GM RZ13 + Holly | 1.66 | 20 | D |
| GM RZ13 | 1.09 | 20 | E |

TABLE 3C
Plants grown in soil from the Pithiviers
area in France infested with P-type BNYVV

| | Mean log ng | Number of | Duncan |
|---|---|---|---|
| Plants of | BNYVV ml⁻¹ | plants | grouping |
| Susceptible Control | 4.28 | 10 | A |
| Conventional Holly 1 | 4.28 | 20 | A |
| Conventional Holly 2 | 3.93 | 20 | A B |
| Conventional Holly 3 | 3.98 | 10 | A B |
| Holly + C48 1 | 3.6 | 20 | B |
| Holly + C48 2 | 2.12 | 20 | C |
| GM RZ13 + Holly | 1.59 | 20 | D |
| GM RZ13 | 1.52 | 20 | D |

The results with the soil from Germany show that plant material of event GM RZ13 also carrying the resistance from Holly is almost free from BNYVV (see Table 3D). As expected, the resistance level is also very high in plant material containing the resistance conferred by the transgenic event GM RZ13 only, but also in plant material from one line (referred to as Holly+C48 2 in Table 3D below) carrying resistances from Holly and C48. Again, plants material from a hybrid obtained from the one line referred to as Holly+C48 2 became significantly more infected by BNYVV than plants of the line itself.

TABLE 3D
Plants grown in soil from Germany infested with B-type BNYVV

| | Mean log ng | Number | Duncan |
|---|---|---|---|
| Plants of | BNYVV ml⁻¹ | of plants | grouping |
| Susceptible Control | 4.37 | 19 | A |
| Conventional Holly 1 | 2.93 | 20 | B |
| Conventional Holly 2 | 2.92 | 20 | B |
| Conventional Holly 3 | 3.18 | 17 | B |
| Holly + C48 1 | 2.94 | 20 | B |
| Holly + C48 2 | 0.92 | 20 | C |
| GM RZ13 + Holly | 0.28 | 20 | D |
| GM RZ13 | 1.02 | 19 | C |

In general, these results show that plant material containing event GM RZ13 alone or in addition to the resistance obtained from Holly as well as plant material from a line combining the conventional resistances from the sources Holly and C48, have the lowest BNYVV content when challenged with BNYVV of different types and origin. However, the good resistance of the line is partially lost in hybrids obtained from crossing this line with other line not containing any resistance (i.e., the hybrid will be heterozygous for the conventional resistance sources); the resulting plants become significantly more susceptible to BNYVV. Plant material containing the event GM RZ 13 showed impressive resistance in all tested soils and was significantly stronger than the resistance in any of the conventional hybrids.

### Example 6

### Field T rials in Sweden

Field trials were performed during the growing seasons in 2004 and 2005 in the South-East of Sweden on a rhizomania-infested field known to carry B-type BNYVV based on the severity of the disease observed in the sugar beet crops of previous years (data not shown). Three different transgenic hybrids (T₂ progenies) derived from R₀ clone 4, one in a background heterozygous for Holly (corresponding to transgenic T₂ hybrid C in Tables 4A and 4B below) and two in a fully susceptible background (corresponding to transgenic T₂ hybrids A and B in the Tables 4A and 4B below), were compared to conventional hybrids heterozygous for Holly, heterozygous for Rizor, C48×Holly, Rizor×Holly, Alba×Holly and to a fully susceptible hybrid. The trials were drilled in April of each year in 3 replicates with 3 rows per plot, 6 m per row. The distance between the plants within a row was 15 cm. At the end of the growing season in September of each year, 5 cm of the very end of individual main root tips were collected, washed and peeled using a potato peeler. Sap was extracted from the epidermic slices that included the root hairs and virus titers were determined by means of ELISA. The same sap was analyzed for PMI activity in order to identify the transgenic and non-transgenic progeny plants. The field trials were executed according to the directives imposed by the Swedish committee for the Experimental release of Genetically Modified Organisms (Jordbruksverket) as outlined in approval number DNR22-6371/03.

When the plants were lifted in the beginning of September, all susceptible controls showed clear rhizomania symptoms. Infected plants were smaller with chlorotic leaves, and the taproots showed the typical abundance of secondary side roots, contrary to the transgenic plants that escaped from infection and remained free of any visual symptoms. The visual observations correlated with the virus titers measured in the roots (Table 4A).

TABLE 4A
T₂ hybrids grown in a Swedish field in 2004
naturally infested with B-type BNYVV

| | Mean log ng | Number of | Duncan |
|---|---|---|---|
| Plants of | BNYVV ml⁻¹ | plants | grouping |
| Rizor × Holly | 1.70 | 59 | A |
| Alba × Holly | 1.59 | 57 | A |
| Rizor × susceptible | 1.57 | 56 | A |
| Holly × susceptible | 1.51 | 60 | A |
| T₂ hybrid A from R₀ clone 4 | 0.89 | 70 | B |
| C48 × Holly | 0.73 | 60 | B C |
| T₂ hybrid B from R₀ | 0.57 | 75 | B C |
| clone 4 | | | |
| T₂ hybrid C from R₀ | 0.33 | 62 | C |
| clone 4 + Holly | | | |

Not included in ANOVA

| | | |
|---|---|---|
| Susceptible control | >2.95 | 50 |

Susceptible controls were highly infected with virus contents of greater than 2.95 log₁₀ ng ml⁻¹. The transgenic hybrids had significantly lower virus content compared to the susceptible controls, but also compared to the resistant hybrids Rizor×Holly, Alba×Holly, heterozygous Rizor and heterozygous Holly. According to Duncan's multiple range test the transgenic hybrids outperformed all resistant checks except for the combination of C48×Holly (statistical group BC) that was not significantly different from the transgenic hybrids (statistical groups B and BC). Interestingly, the hybrid heterozygous for both Holly and the transgenic resistance (statistical group C) showed the lowest virus titers of all entries, although the difference was not always significant. Nevertheless, this observation illustrates the potential of combining transgenic and conventional resistance sources so as to obtain yet superior resistance to rhizomania.

Similar results have been obtained in the field trial in 2005 as can be seen in Table 4B. The transgenic plants also carrying the Holly resistance had significantly lower BNYVV content compared to all other tested materials. The two Holly×C48 hybrids had significantly lower virus content compared to pure Holly materials.

TABLE 4B
T₂ hybrids grown in a Swedish field in 2005
naturally infested with B-type BNYVV

| | Mean log ng | Number of | Duncan |
|---|---|---|---|
| Plants of | BNYVV ml⁻¹ | plants | grouping |
| Susceptible hybrid | 2.58 | 39 | B |
| Conventional Holly hybrid | 1.87 | 40 | C |
| Conventional Holly hybrid | 1.50 | 79 | C D |
| Conventional Holly + | 1.20 | 40 | D E |
| C48 hybrid | | | |
| Conventional Holly + | 0.84 | 40 | E |
| C48 hybrid | | | |
| Conventional Holly + | 0.77 | 40 | E |
| C48 hybrid | | | |
| GM RZ 13 + conventional | 0.28 | 40 | F |
| Holly hybrid | | | |

Not included in ANOVA

| | | |
|---|---|---|
| Susceptible control | >2.95 | 50 |

Thus, in both field trials in Sweden the transgenic materials contained significantly less BNYVV than all conventional resistant hybrids except for hybrids based on a combination of Holly and C48.

### Example 7

### Field Trials in the USA

A further field trial was performed during the growing seasons in 2009 on a rhizomania-infested field in Raymond, Minn., USA, known to carry a deviant resistant-breaking strain of the A-type BNYVV. Sugar beet seed of transgenic event GM RZ13 crossed with a conventional line carrying the “Holly” resistance was treated with standard fungicides according to the manufacturer's recommendations [Apron (metalaxyl, Syngenta, Greensboro, N.C.) and Thiram (tetramethylthiuram disulfide, Bayer CropScience, Research Triangle Park, N.C.)] and were planted in a randomized block design (6 replications/entry). Each replicate was represented by a single plot of 10 m² (110 square feet) with 3 rows spaced about 56 cm (22 inches) apart, planted to stand with a 13 cm (5 inch) seed spacing using a John Deere air planter. Appropriate agronomic practices were employed to maintain adequate plant health, including microrate herbicide and pesticide application and cultivation. Approximately 14 weeks post planting, the beets were topped; individual plots were dug and bagged using a research grade sugar beet harvester. Each plot was individually processed using a sugar beet tare line (Relobo, Parma, Italy). Following automated washing, weighing and slicing of the beets by the tare line, four 30 gram samples of sugar beet brei was automatically extruded and collected for sugar and impurity analysis using an automated Venema beet analyzing system (Venema, Groningen, Netherlands).

The brei samples were used for sugar analysis and for quantification of the BNYVV content by ELISA. Virus titers were determined by means of ELISA according to the method of Clark et al. (1977) and Gidner et al. (2005). For the ELISA, 0.2 g brei per sample were diluted and properly mixed in PBS-Tween-albumine extraction buffer (1:20 w/v; extraction buffer is as described in the references for the ELISA method).

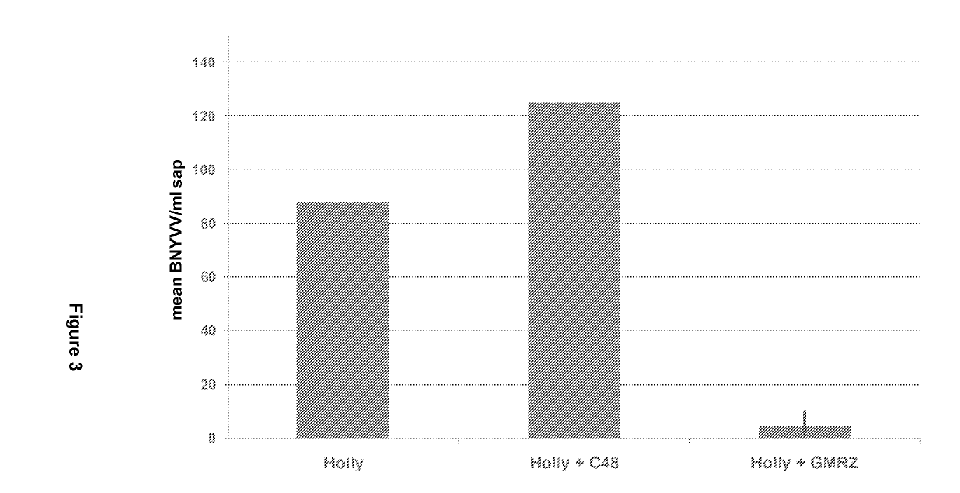

Plants carrying conventional resistances against Rhizomania were used as controls in the field trials in 2009. The control plants carrying the resistance from Holly (“Holly”) or from Holly and C48 (“Holly+C48”), respectively, were highly infected with BNYVV compared to the transgenic plants carrying the GM RZ13 event in combination with the conventional resistance from Holly (“Holly”; see FIG. 3). The transgenic plants carrying the GM RZ13 event clearly showed the lowest virus contents compared to the control with the conventional resistance from Holly only, but also compared to the plants with the combined conventional resistances from Holly and C48 (FIG. 3).

### Example 8

### Summary or Results of the Trials in a Climate Chamber and in the Fields

The sugar beet plants of the present invention containing event GM RZ13 alone showed superior resistance compared with the native sources as shown in Table 4. The tested material have been plant material containing event GM RZ13 (“GM RZ13”), plant material containing both conventional resistances from Holly and C48 (“Holly+C48”), plant material containing the conventional resistance from Holly (“Holly”), and plant material containing neither the transgenic event nor a conventional resistance (“Susceptible”).

TABLE 5
Summary of the results of the field trials with different
types of BNYVV discussed in Examples 5 to 7 above

| Description of soil | | Plant material tested | | | |
|---|---|---|---|---|---|
| Virus | | | | | Suscep- |
| type | | GM RZ13 | Holly + C48 | Holly | tible |
| A | Medium to high | +++ | ++ | + | − |
| | infection pressure; | | | | |
| | Soil from US, | | | | |
| | Spain and Iran | | | | |
| A | High infection/ | +++ | + | − | − |
| | deviant virus | | | | |
| | strains; Soil | | | | |
| | from US and Spain | | | | |
| | strains; Soil from | | | | |
| | US and Spain | | | | |
| B | Medium to high | +++ | ++ | + | − |
| | infection pressure; | | | | |
| | Soil from Germany | | | | |
| | and Sweden | | | | |
| P | Medium to high | +++ | ++ | + | − |
| | infection pressure; | | | | |
| | Soil from France | | | | |
| | (Pithiviers) | | | | |
| P | Very high | +++ | + | − | − |
| | infection pressure; | | | | |
| | Soil from France | | | | |
| | (Pithiviers) | | | | |

Control scale:
+++ Extremely low virus content
++ medium virus content
+ high virus content
− very high virus content/susceptible level

As can be seen from Table 5, plants containing event GM RZ13 show a consistently and strong reduction of the virus titer of all types of BNYVV compared to conventional resistances. Further, the plants containing event GM RZ13 of the present invention also shows a strong control of a new highly pathogenic BNYVV strain against, the multiplication of which is not or just partially reduced by the conventional resistances.

### DEPOSIT

Applicants have made a deposit of sugar beet seed of event GM RZ13 disclosed above on Dec. 11, 2008 in accordance with the Budapest Treaty at the NCIMB Ltd. Ferguson Building, Craibstone Estate, Bucksbum, Aberdeen AB21 9YA, Scotland under NCIMB Accession No. NCIMB 41601. The deposit will be maintained in the depositary for a period of 30 years, or 5 years after the last request, or the effective life of the patent, whichever is longer, and will be replaced as necessary during that period. Applicants impose no restrictions on the availability of the deposited material from the ATCC; however, Applicants have no authority to waive any restrictions imposed by law on the transfer of biological material or its transportation in commerce. Applicants do not waive any infringement of their rights granted under this patent or under the Plant Variety Protection Act (7 USC 2321 et seq.).

All publications and published patent documents cited in this specification are incorporated herein by reference to the same extent as if each individual publication or patent document was specifically and individually indicated to be incorporated by reference.

### REFERENCES

Abe H, Tamada T (1986) Association of Beet Necrotic Yellow Vein Virus with isolates of Polymyxa betae Keskin. Ann Phytopath Soc Jpn 52:235-247
Baulcombe D C (1996) Mechanisms of pathogen-derived resistance to viruses in transgenic plants. Plant Cell 8:1833-1844
Biancardi E, Lewellen R T, De Biaggi M, Erichsen A W, Stevanato P (2002) The origin of rhizomania resistance in sugar beet. Euphytica 127:383-397
Bouzoubaa S, Quillet L, Guilley H, Jonard G, Richards K (1987) Nucleotide sequence of Beet Necrotic Yellow Vein Virus RNA-1. J Gen Virol 68:615-626
Canova A (1959) Appunti di patologia della barbabietola. Informatore Fitopatologico 9:390-396
Clark M F, Adams A N (1977) Characteristics of the microplate method of enzyme-linked immunosorbent assay for the detection of plant viruses. J Gen Virol 34:475-483
De Biaggi M (1987) Me'thodes de se'lection—un cas concret. Proceedings of the 50th IIRB winter congress, Brussels, Vol.II, pp 157-163
Gidner S, Lennefors B-L, Nilsson N-O, Bensefelt J, Johansson E, Gyllenspetz U, Kraft T (2005) QTL mapping of BNYVV resistance from the WB41 source in sugar beet. Genome 48:279-285
Joersbo M, Donaldson I, Kreiberg J, Petersen S G, Brunstedt J, Okkels F T (1998) Analysis of mannose selection used for transformation of sugar beet. Mol Breed 4:111-117
Johansen L K, Carrington J C (2001) Silencing on the spot Induction and suppression of RNA silencing in the Agrobacterium-mediated transient expression system. Plant Physiol 126:930-938
Kaniewski W, Lawson C (1998) Coat protein and replicase-mediated resistance to plant viruses. In: Hadidi A, Khetarpal R K, Koganezawa H (eds) Plant virus disease control. APS PRESS, Saint-Paul, Minn., USA, pp 65-78
Koenig R, Lü ddecke P, Haeberle' A M (1995) Detection of Beet Necrotic Yellow Vein Virus strains, variants and mixed infections by examining single-strand conformation polymorphisms of immunocapture RT-PCR products. J Gen Virol 76:2051-2055
Kreuze J F, Savenkov E l, Cuellar W, Li X, Valkonen J P T (2005) Viral class 1 RNase III involved in suppression of RNA silencing. J Virol 79:7227-7238
Kruse M, Koenig R, Hoffmann A, Kaufmann A, Commandeur U, Solovyev A G, Savenkov I, Burgermeister W (1994) Restriction fragment length polymorphism analysis of reverse transcription-PCR products reveals the existence of two major strain groups of Beet Necrotic Yellow Vein Virus. J Gen Virol 75:1835-1842
Lennefors B-L, Savenkov E l, Mukasa S B, Valkonen J P T (2005) Sequence divergence of four Soilborne Sugar Beet-infecting Viruses. Virus Genes 31:57-64
Lennefors B-L, Sarenkov E l, Bensefeld J, Wremerth-Weich E, van Roggen P, Tuvesson S, Valkonen J P T, Gielen J (2006) dsRNA-mediated resistance to Beet Necrotic Yellow Vein Virus infection in sugar beet (Beta vulgaris L. ssp. vulgaris). Mol Breeding 18: 313-325.
Lewellen R T, Skoyen I O, Erichsen A W (1987) Breeding sugar beet for resistance to rhizomania: Evaluation of host-plant reactions and selection for and inheritance of resistance. Proceedings of the 50th IIRB winter congress, Brussels, Vol. II, pp 139-156
Lewellen R T, Whitney E D (1993) Registration of germplasm lines developed from composite crosses of sugarbeet×Beta maritima. Crop Sci 33:882-883
Lewellen R T (1995) Performance of near-isolines of sugarbeet with resistance to rhizomania from different sources. In: Proc 58th IIRB cong: 83-92
Liu H-Y, Sears J L, Lewellen R T (2005) Occurrence of resistance-breaking Beet Necrotic Yellow Vein Virus of sugar beet. Plant Dis 89:464-468
Murashige T, Skoog F (1962) A revised medium for rapid growth and bioassays with tobacco tissue cultures. Physiol Plant 15:473-497
Norris S R, Meyer S E, Callis J (1993) The intron of Arabidopsis thaliana polyubiquitin genes is conserved in location and is a quantitative determinant of chimeric gene expression. Plant Mol Biol 21:895-906
Paul H, Henken B, Alderlieste M F J (1992) A greenhouse test for screening sugar beet (Beta vulgaris) for resistance to Beet Necrotic Yellow Vein Virus (BNYVV). Neth J Plant Pathol 98:65-75
Pelsy F, Merdinoglu D (1996) Identification and mapping of random amplified polymorphic DNA markers linked to a rhizomania resistance gene in sugar beet (Beta vulgaris L.) by bulked segregant analysis. Plant Breeding 115:371-377
Powell-Abel P, Nelson R S, De B, Hoffmann N, Rogers S G, Fraley R T, Beachy R N (1986) Delay of disease development in transgenic plants that express the Tobacco Mosaic Virus coat protein gene. Science 232:738-743
Reed J, Privalle L, Powell M L, Meghji M, Dawson J, Dunder E, Suttie J, Wenck A, Launis K, Kramer C, Chang Y F, Hansen G, Wright M (2001) Phosphomannose isomerase: an efficient selectable marker for plant transformation. In Vitro Cell Dev Biol Plant 37:127-132
Sanford J C, Johnston S A (1985) The concept of parasitederived resistance—Deriving resistance genes from parasite's own genome. J Theor Biol 113:395-405
Scholten O E, Jansen R C, Keizer L C P, De Bock T S M, Lange W (1996) Major genes for resistance to Beet Necrotic Yellow Vein Virus (BNYVV) in Beta vulgaris. Euphytica 91:331-339
Tamada T, Baba T (1973) Beet Necrotic Yellow Vein Virus from rhizomania-affected sugar beet in Japan. Ann Phytopathol Soc Jpn 39:325-332

1-38. (canceled)

39. An isolated nucleic acid molecule, wherein the nucleotide sequence of the nucleic acid, molecule comprises a nucleic acid sequence selected from the group consisting of SEQ ID NO: 1, SEQ ID NO: 2, SEQ ID NO: 7, SEQ ID NO: 8, and the complements thereof.

40. The isolated nucleic acid molecule of claim 39, wherein the nucleic acid molecule is comprised in a sugar beet seed deposited at NCIMB under the accession No. 41601.

41. A pair of polynucleotide primers comprising a first polynucleotide primer and a second polynucleotide primer which function together in the presence of a sugar beet event GM RZ13 DNA template to produce an amplicon diagnostic for the sugar beet event GM RZ13.

42. The pair of polynucleotide primers of claim 41, wherein the first polynucleotide primer is or is complementary to a sugar beet plant genome sequence flanking the point of insertion of a heterologous DNA sequence inserted into the sugar beet plant genome of sugar beet event GM RZ13, and wherein the second polynucleotide primer is or is complementary to the heterologous DNA sequence inserted into the sugar beet plant genome of the sugar beet event GM RZ13.

43. The pair of primers according to claim 41, wherein the first or second polynucleotide primer is selected from the group consisting of SEQ ID NO: 2, SEQ ID NO: 3, SEQ ID NO: 8 or SEQ ID NO: 9.

44. The pair of polynucleotide primers of claim 41, wherein the first polynucleotide primer is a primer selected from the group consisting of
a. a polynucleotide primer comprising at least 10 contiguous nucleotides from SEQ ID NO: 3 or from position 461-807 as set forth as SEQ ID NO: 2, or the complements thereof; and
b. a polynucleotide primer comprising at least 10 contiguous nucleotides from SEQ ID NO: 9 or from position 1-237 as set forth as SEQ ID NO: 8, or the complements thereof.

45. The pair of polynucleotide primers according to claim 41, wherein the first polynucleotide primer comprises a nucleotide sequence selected from the group consisting of SEQ ID NO: 12, SEQ ID NO: 13, SEQ ID NO: 14, SEQ ID NO:15, SEQ ID NO:16, SEQ ID NO:23, SEQ ID NO:24, SEQ ID NO:25, and the complements thereof.

46. The pair of polynucleotide primers according claim 41, wherein the second polynucleotide primer is a primer selected from the group consisting of
a. a polynucleotide primer comprising at least 10 contiguous nucleotides from position 1-460 as set forth as SEQ ID NO: 2, or the complements thereof; and
b. a polynucleotide primer comprising at least 10 contiguous nucleotides from position 238-484 as set forth as SEQ ID NO: 8, or the complements thereof.

47. The pair of polynucleotide primers of claim 41, wherein the second polynucleotide primer comprises a nucleotide sequence selected from the group consisting of SEQ ID NO: 4, SEQ ID NO: 5, SEQ ID NO: 6, SEQ ID NO: 10, SEQ ID NO: 18, SEQ ID NO: 19, SEQ ID NO: 20, SEQ ID NO: 26, and the complements thereof.

48. The pair of polynucleotide primers of claim 41, wherein the pair of primers is selected from the group of primer pairs consisting of:
a. the polynucleotide primer as set forth as SEQ ID NO: 13 as the first polynucleotide primer and the second polynucleotide primer as set forth as SEQ ID NO: 18, and complements thereof;
b. the polynucleotide primer as set forth as SEQ ID NO: 14 as the first polynucleotide primer and the second polynucleotide primer as set forth as either SEQ ID NO: 10 or SEQ ID NO: 18, and complements thereof;
c. the polynucleotide primer as set forth as SEQ ID NO: 15 as the first polynucleotide primer and the second polynucleotide primer as set forth as SEQ ID NO: 18, and complements thereof;
d. the polynucleotide primer as set forth as SEQ ID NO: 16 as the first polynucleotide primer and the second polynucleotide primer as set forth as SEQ ID NO: 18, and complements thereof;
e. the polynucleotide primer as set forth as SEQ ID NO: 12 as the first polynucleotide primer and the second polynucleotide primer as set forth as either SEQ ID NO: 19, SEQ ID NO: 4, SEQ ID NO: 5, or SEQ ID NO: 6, and complements thereof;
f. the polynucleotide primer as set forth as SEQ ID NO: 19 as the first polynucleotide primer and the second polynucleotide primer as set forth as SEQ ID NO: 24, and complements thereof;
g. the polynucleotide primer as set forth as SEQ ID NO: 20 as the first polynucleotide primer and the second polynucleotide primer as set forth as SEQ ID NO: 24, and complements thereof; and
h. the polynucleotide primer as set forth as SEQ ID NO: 25 as the first polynucleotide primer and the second polynucleotide primer as set forth as SEQ ID NO: 26, and complements thereof.

49. The pair of polynucleotide primers of claim 41, wherein the pair of primers is selected from the group of primer pairs consisting of:
a) the polynucleotide primer as set forth as SEQ ID NO: 13 as the first polynucleotide primer and the second polynucleotide primer as set forth as either SEQ ID NO: 11 or SEQ ID NO: 17, and complements thereof;
b) the polynucleotide primer as set forth as SEQ ID NO: 12 as the first polynucleotide primer and the second polynucleotide primer as set forth as either SEQ ID NO: 21 or SEQ ID NO: 22, and complements thereof;
c) the polynucleotide primer as set forth as SEQ ID NO: 19 as the first polynucleotide primer and the second polynucleotide primer as set forth as SEQ ID NO: 23, and complements thereof; and
d) the polynucleotide primer as set forth as SEQ ID NO: 20 as the first polynucleotide primer and the second polynucleotide primer as set forth as SEQ ID NO: 23, and complements thereof.

50. A method of detecting the presence of a nucleic acid molecule comprising the steps of:
a. collecting a DNA sample from sugar beet event GM RZ13;
b. contacting the DNA sample with a pair of primers under conditions appropriate for nucleic acid amplification and thereby producing an amplicon that is diagnostic for sugar beet event GM RZ13; and
c. detecting the amplicon.

51. The method of claim 50, wherein the pair of primers is a first polynucleotide primer and a second polynucleotide primer, wherein the first polynucleotide primer is or is complementary to a sugar beet plant genome sequence flanking the point of insertion of a heterologous DNA sequence inserted into the sugar beet plant genome of sugar beet event GM RZ13, and wherein the second polynucleotide primer is or is complementary to the heterologous DNA sequence inserted into the sugar beet plant genome of the sugar beet event GM RZ13.

52. The method of claim 50, wherein said pair of polynucleotide primers is selected from the group consisting of the pair of primers depicted by SEQ ID NO: 5 and 12, and the pair of primers depicted by SEQ ID NO: 13 and 18; and wherein the detecting the amplicon is performed by a gel based assay.

53. The method of claim 50, wherein said pair of polynucleotide primers is depicted by SEQ ID NOs: 25 and 26 and further comprises a probe having the sequence as set forth as SEQ ID NO: 27.

54. A method of detecting the presence of a nucleic acid molecule comprising the steps of:
a. collecting a DNA sample from a sugar beet event GM RZ13;
b. contacting the sample with a probe that hybridizes under high stringency conditions with genomic DNA from event GM RZ13 and does not hybridize under high stringency conditions with DNA of a control sugar beet plant;
c. subjecting the sample and probe to high stringency hybridization conditions; and
d. detecting hybridization of the probe to the DNA sample from event GM RZ13.

55. The method of claim 54, wherein said probe comprises a nucleotide sequence selected from the group consisting of SEQ ID NO: 1, SEQ ID NO: 2, SEQ ID NO: 3, SEQ ID NO: 7, SEQ ID NO: 8, SEQ ID NO: 9, and complements thereof.

56. A kit for detecting the presence of nucleic acids that are unique to sugar beet event GM RZ13 in a biological sample, the kit comprising at least one nucleic acid molecule of sufficient length of contiguous polynucleotides to function as a primer or probe in a nucleic acid detection method, and which upon amplification of or hybridization to a target nucleic acid sequence in a sample followed by detection of the amplicon or hybridization to the target sequence, are diagnostic for the presence of nucleic acid sequences unique to sugar beet event GM RZ13 in the sample.

57. The kit of claim 56, wherein the nucleic acid molecule of sufficient length of contiguous polynucleotides to function as a primer or probe is selected from the group consisting of SEQ ID NO: 1, SEQ ID NO: 4, SEQ ID NO: 5, SEQ ID NO: 6, SEQ ID NO: 7, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and complements thereof.

58. A transgenic Beet Necrotic Yellow Vein Virus resistant sugar beet plant, cells or tissues thereof, comprising the nucleic acid molecule of claim 39.

59. The transgenic Beet Necrotic Yellow Vein Virus resistant sugar beet plant of claim 58, wherein seed of said plant is deposited under NCIMB Accession No: 41601.

60. A plant produced from the transgenic Beet Necrotic Yellow Vein Virus resistant sugar beet seed of claim 59.

61. A sugar beet seed comprising the nucleic acid molecule of claim 39.

62. The sugar beet seed of claim 61, wherein said seed has been deposited at the NCIMB under NCIMB accession number 41601.

63. The transgenic necrotic yellow vein virus resistant sugar beet plant producted by the seed according of claim 61.

64. A biological sample or an extract from the sugar beet event GM RZ13, wherein said sample or said extract comprises a nucleotide sequence that is or is complementary to a nucleotide sequence selected from the group consisting of SEQ ID NO: 1, SEQ ID NO: 2, SEQ ID NO: 7 and SEQ ID NO: 8, and wherein the sequence is detectable in the sample using a nucleic acid amplification or nucleic acid hybridization method.

65. A method for producing a sugar beet plant resistant to at least Beet Necrotic Yellow Vein Virus, the method comprising the steps of
a. sexually crossing a first parent sugar beet plant with a second parent sugar beet plant, wherein said first or second parent sugar beet plant comprises sugar beet event GM RZ13 DNA, thereby producing a plurality of first generation progeny plants;
b. selecting a first generation progeny plant that is resistant to at least Beet Necrotic Yellow Vein Virus;
c. selfing the first generation progeny plant, thereby producing a plurality of second generation progeny plants;
d. selecting from the second generation progeny plants, a plant that is at least resistant to Beet Necrotic Yellow Vein Virus;
wherein the second generation progeny plants comprise a nucleotide sequence that is or is complementary to a nucleotide sequence selected from the group consisting of SEQ ID NO: 1 and SEQ ID NO: 7.

66. Method according to claim 65, wherein said first or second parent sugar beet plant comprising sugar beet event GM RZ13 DNA in step a) is the Beet Necrotic Yellow Vein Virus resistant sugar beet plant produced by the seeds deposited at the NCIMB under NCIMB accession number 41601.

67. A method of producing Beet Necrotic Yellow Vein Virus resistant sugar beet hybrid seeds, the method comprising the steps of:
a. providing a Beet Necrotic Yellow Vein Virus resistant sugar beet line as a first parent line;
b. providing a second sugar beet line having a different genotype than the first parent line as a second parent line;
wherein one of the parent lines of step a) or step b) is a male sterile CMS line and wherein the other parent line is male fertile, and
c. allowing the plants of the male fertile parent line to pollinate the flowers of the male sterile parent line, allowing the seed to develop, and harvesting the hybrid seed;
wherein the harvested hybrid seeds are seeds of a Beet Necrotic Yellow Vein Virus resistant sugar beet hybrid plant.

68. A method of producing Beet Necrotic Yellow Vein Virus resistant sugar beet hybrid seeds of claim 67, wherein the male sterile CMS sugar beet parental line provided in step a) or b) is an inbred sugar beet line comprising a nucleotide sequence selected from the group consisting of SEQ ID NO: 1, SEQ ID NO: 2, SEQ ID NO: 7, SEQ ID NO: 8, and the complements thereof.

69. A method of producing Beet Necrotic Yellow Vein Virus resistant sugar beet hybrid seeds of claim 67, wherein the second parental line is selected from the group consisting of
a. an inbred sugar beet plant line resistant to at least Beet Necrotic Yellow Vein Virus having a different genotype than the first parental line wherein the inbred sugar beet plant comprises a nucleic acid sequence selected from the group consisting of SEQ ID NO: 1, SEQ ID NO: 2, SEQ ID NO: 7, SEQ ID NO: 8, and the complements thereof;
b. an inbred sugar beet plant line resistant or tolerant to at least Beet Necrotic Yellow Vein Virus which originates from a naturally occurring source selected from the group comprising the Holly source, WB41, WB42, WB151, WB169, C28, C48, C50, or Rizor, or crosses thereof; and
c. an inbred sugar beet plant line having no resistance or tolerance to the Beet Necrotic Yellow Vein Virus.

70. Hybrid seed of a transgenic Beet Necrotic Yellow Vein Virus resistant sugar beet.

71. Hybrid seed, wherein the seed is produced by the method of claim 65.

72. A hybrid Beet Necrotic Yellow Vein Virus resistant sugar beet plant produced by growing the hybrid seed of claim 70.

73. A method for producing sugar or one or more biofuel(s) selected from the group comprising ethanol, butanol, biogas and/or biodiesel, comprising the steps of: a) providing a sugar beet plant or plant part of sugar beet event GM RZ13; and b) processing said sugar beet plant or plant to produce sugar or one or more biofuels.


SEQUENCE LISTING (30 sequences)

SEQ ID NO: 1 - DNA, 20 bp, Artificial Sequence
  3′ junction sequence between RZ inset and genomic DNA of sugar beet
acaccacaatttataccaac

SEQ ID NO: 2 - DNA, 807 bp, Artificial Sequence
  Sequence spanning 3′ end of insert and flanking sugar beet genomic DNA
ggtgaatcagcgtttattgccgccaacgaatcaccggtgactgtcaaaggccacggccgtttagcgcgtgtttacaacaagctgtaagagcttactgaaaaaattaacatctcttgctaagctgggagctcgtcgacgcatgcccgctgaaatcaccagtctctctctacaaatctatctctctctataataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccaggggtacgatctcgagagatctgctagccctgcaggaaatttaccggtgcccgggcggccagcatggccgtatccgcaatgtgttattaagttgtctaagcgtcaatttgtttacaccacaatttataccaaccaaacaacacttaacatgagaggaggaactggtggtgctatggtggaagagaggtaggagaaagaaaaaaaatttataccaaccaaacaacacttaacatctaagggttttgcttccctttccctctctacccctttcttgattccattccctttaccccttgacaaccaaacgccccctcagttctcatgagttaggaagtaagaaggagattctaacacataagaaaacttccaaagacggaaagtggtgaatttatacctagaagcaaatattcacgccaataaataccccaaatatgaggatgatgagagtcgaactcccgaccataaggttg

SEQ ID NO: 3 - DNA, 347 bp, Beta vulgaris
ttataccaaccaaacaacacttaacatgagaggaggaactggtggtgctatggtggaagagaggtaggagaaagaaaaaaaatttataccaaccaaacaacacttaacatctaagggttttgcttccctttccctctctacccctttcttgattccattccctttaccccttgacaaccaaacgccccctcagttctcatgagttaggaagtaagaaggagattctaacacataagaaaacttccaaagacggaaagtggtgaatttatacctagaagcaaatattcacgccaataaataccccaaatatgaggatgatgagagtcgaactcccgaccataaggttg

SEQ ID NO: 4 - DNA, 18 bp, Artificial Sequence
  Polynucleotide sequence
cggccagcatggccgtat

SEQ ID NO: 5 - DNA, 22 bp, Artificial Sequence
  Polynucleotide sequence
ccagcatggccgtatccgcaat

SEQ ID NO: 6 - DNA, 24 bp, Artificial Sequence
  Polynucleotide sequence
catggccgtatccgcaatgtgtta

SEQ ID NO: 7 - DNA, 20 bp, Artificial Sequence
  5′ junction sequence between RZ inset and genomic DNA of sugar beet
cggatgcgactcaaacactg

SEQ ID NO: 8 - DNA, 484 bp, Artificial Sequence
  Sequence spanning 5′ end of insert and flanking sugar beet genomic DNA
gatcaccgaaaccctaacccgaccaccgaaaccctaacccaaccaccgtttgaagctctgtttttacagacatgctggcggaaccctaactcacgacgctattggatgggcggacgcgacaacaggggaggagagtggagaagaatacgagggtggcgggcgggcggatgcgacgcaggggagggggtgcgagaagacgcgacgcaggggagagggtggcgggcgggcggatgcgactcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcccgatctagtaacatagatgacaccgcgcgcgataatttatcctagttgcgcgctata

SEQ ID NO: 9 - DNA, 237 bp, Beta vulgaris
gatcaccgaaaccctaacccgaccaccgaaaccctaacccaaccaccgtttgaagctctgtttttacagacatgctggcggaaccctaactcacgacgctattggatgggcggacgcgacaacaggggaggagagtggagaagaatacgagggtggcgggcgggcggatgcgacgcaggggagggggtgcgagaagacgcgacgcaggggagagggtggcgggcgggcggatgcgac

SEQ ID NO: 10 - DNA, 26 bp, Artificial Sequence
  Polynucleotide sequence
cagcgttgcggttctgtcagttccaa

SEQ ID NO: 11 - DNA, 32 bp, Artificial Sequence
  Polynucleotide sequence
atgagatgggtttttatgattagagtcccgca

SEQ ID NO: 12 - DNA, 27 bp, Artificial Sequence
  Polynucleotide sequence
tcgactctcatcatcctcatatttggg

SEQ ID NO: 13 - DNA, 22 bp, Artificial
  Polynucleotide sequence
agaagacgcgacgcaggggaga

SEQ ID NO: 14 - DNA, 27 bp, Artificial
  Polynucleotide sequence
agacatgctggcggaaccctaactcac

SEQ ID NO: 15 - DNA, 27 bp, Artificial
  Polynucleotide sequence
cctaactcacgacgctattggatgggc

SEQ ID NO: 16 - DNA, 27 bp, Artificial
  Polynucleotide sequence
gcgacaacaggggaggagagtggagaa

SEQ ID NO: 17 - DNA, 29 bp, Artificial
  Polynucleotide sequence
gacctaatccaaccaccaccataggatgt

SEQ ID NO: 18 - DNA, 24 bp, Artificial
  Polynucleotide sequence
tgcagcgttgcggttctgtcagtt

SEQ ID NO: 19 - DNA, 31 bp, Artificial
  Polynucleotide sequence
ggtgaatcagcgtttattgccgccaacgaat

SEQ ID NO: 20 - DNA, 20 bp, Artificial
  Polynucleotide sequence
gccgtatccgcaatgtgtta

SEQ ID NO: 21 - DNA, 26 bp, Artificial
  Polynucleotide sequence
gatgggcgcacatccgaaaagcagtt

SEQ ID NO: 22 - DNA, 23 bp, Artificial
  Polynucleotide sequence
gcatgcaagcttcgtacgttaat

SEQ ID NO: 23 - DNA, 27 bp, Artificial
  Polynucleotide sequence
cgaccctcacaaagacaaagaaatatc

SEQ ID NO: 24 - DNA, 22 bp, Artificial
  Polynucleotide sequence
caaccttatggtcgggagttcg

SEQ ID NO: 25 - DNA, 17 bp, Artificial
  polynucleotide primer
gggcggatgcgactcaa

SEQ ID NO: 26 - DNA, 23 bp, Artificial
  Polynucleotide sequence
ccttaattctccgctcatgatca

SEQ ID NO: 27 - DNA, 15 bp, Artificial
  Polynucleotide sequence - TaqMan probe
cgggaaacgacaatc

SEQ ID NO: 28 - DNA, 29 bp, Artificial
  Polynucleotide sequence
tcgtagaagagaattcacccaaactatcc

SEQ ID NO: 29 - DNA, 29 bp, Artificial
  Polynucleotide sequence
aagaattgcaggatccacaggctcggtac

SEQ ID NO: 30 - DNA, 24 bp, Artificial
  Polynucleotide sequence
ttccaacgaattcggtctcagaca